(19) United States
(12) Patent Application Publication
(10) Pub. No.: US 2026/0327617 A1
(43) Pub. Date: Oct. 22, 2026

(54) OLIGONUCLEOTIDE

(71) Applicant: ACADEMISCH ZIEKENHUIS LEIDEN H.O.D.N. LUMC, Leiden (NL)

(72) Inventors: Anton Jan VAN ZONNEVELD, Soest (NL); Jurriën PRINS, Oegstgeest (NL); Eric Peter VAN DER VEER, The Hague (NL); Bianca MATTHEE, Amsterdam (NL)

(21) Appl. No.: 18/862,676

(22) PCT Filed: May 5, 2023
(86) PCT No.: PCT/IB2023/054702

§ 371 Date: Nov. 4, 2024

(30) Foreign Application Priority Data
May 6, 2022 (EP) 22172149.1

(51) Int. Cl. C12N 15/113; A61P 31/20

(52) U.S. Cl. CPC C12N 15/1131; A61P 31/20; C12N 2310/10; C12N 2310/315

(57) ABSTRACT

The invention relates to the field of oligonucleotides binding to a polyomavirus RNA. Such oligonucleotides may be used for the treatment of any disease or condition caused by or associated with such virus.

### RELATED APPLICATIONS

This application is a 35 U.S.C. § 371 filing of International Patent Application No. PCT/IB2023/054702, filed May 5, 2023, which claims priority to European Patent Application No. 22172149.1, filed May 6, 2022, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Jun. 16, 2026, is named 758866_LUMC9-003US_ST26.xml and is 176,895 bytes in size.

### FIELD

The invention relates to the field of oligonucleotides binding to polyomavirus RNA. Such oligonucleotides may be used for the treatment of any disease or condition caused by or associated with such virus.

### BACKGROUND OF THE INVENTION

Polyomaviruses are small non-enveloped double-stranded DNA viruses whose natural hosts are normally mammals and birds. Infections in adults are mostly asymptomatic but can become pathological when the immune system is compromised. Examples of human polyomaviruses are BK virus (BKV), JC virus (JCV) and Merkel cell virus (MCV).

JCV and BKV are both opportunistic pathogens which infect humans during early childhood (Leploeg, M. D. et. al., Clinical Infectious Diseases, 2001). The seroprevalence in adults is high. Both viruses are thought to remain latent in kidney cells of the host (Wunderink, H. F. et. al., American Journal of Transplantation, 2017). Reactivation can occur, for instance, in immunosuppressed individuals (Wunderink, H. F. et. al., American Journal of Transplantation, 2017; Parajuli, S. et. al., Clinical Transplantation, 2018; Gard, L. et. al., PLoS One, 2017).

Polyomaviruses share a common genome structure. They have genes that are expressed both early and late in the infection cycle. Both early and late genes produce RNAs from which, through differential splicing, various proteins can be translated. The late genes typically encode the three capsid proteins, whereas the early genes encode the small and large T-antigens and often include one or more alternatively spliced coding regions (Helle, F. et. al., Viruses, (2017).3; 9(17): 327, 1-18).

WO2019/168402 describes antisense oligonucleotides to modulate splicing of a polyomavirus large T-antigen pre-mRNA. Such antisense oligonucleotides may have a sequence that is complementary to a splice donor site and/or a splice acceptor site in said pre-mRNA.

However, there is still a need for new and improved antisense oligonucleotides for the treatment for diseases or conditions caused by or associated with polyomavirus.

### SUMMARY OF THE INVENTION

In a first aspect, the invention relates to an oligonucleotide comprising a nucleobase sequence according to one of SEQ ID NOs: 1 to 11 or comprising a nucleobase sequence which is analogous to any one of SEQ ID NOs: 1 to 11 characterized in that at least one nucleobase of said SEQ ID NOs is replaced by a nucleobase analogue having the same base pairing specificity as the replaced nucleobase.

In a second aspect, the invention relates to a vector comprising (i) an oligonucleotide as defined in the first aspect, (ii) the reverse complement of an oligonucleotide as defined in the first aspect, or (iii) DNA capable of being transcribed to an oligonucleotide as defined in the first aspect.

In a third aspect, the invention relates to a pharmaceutical composition comprising an oligonucleotide as defined in the first aspect or a vector as defined in the second aspect.

In a fourth aspect, the invention relates to an oligonucleotide as defined in the first aspect, a vector as defined in the second aspect or a pharmaceutical composition as defined in the third aspect for use as a medicament, in particular for use in treating a polyomavirus infection in a subject.

In a fifth aspect, the invention relates to ex-vivo methods, including an ex-vivo method of inhibiting polyomavirus replication in a cell, and an ex-vivo method of producing a transplant, both comprising the use of an oligonucleotide as defined in the first aspect, a vector as defined in the second aspect or a pharmaceutical composition as defined in the third aspect.

### DESCRIPTION OF THE FIGURES

FIG. 1: BKV T-Ag splice sites are conserved between genotypes, allowing for a “one-size-fits-all” ASO approach. Publicly available complete genomic sequences of BKV isolates were downloaded and whole-gene T-Ag sequences were aligned and studied for between-genotype conservation. Genotypes were identified using reference isolates described in literature. Findings reveal that the donor splice site in T-Ag is predominantly conserved between genotypes, while the acceptor splice site is more variable in the non-coding intronic region. Based on these findings, we identified a stretch of nucleotides overlapping the donor splice site that shows a 100% sequence conservation between genotypes. Condensed BKV T-Ag phylogenetic tree (left) with sequence logos (right) depicting the splice site conservation per genotype. The size of the letter (nucleotide) indicates the relative occurrence of the nucleotide being present on that position for every genotype. Deletions are depicted as blank spaces or gaps in the sequence logo. The sequences are given in Table 1.

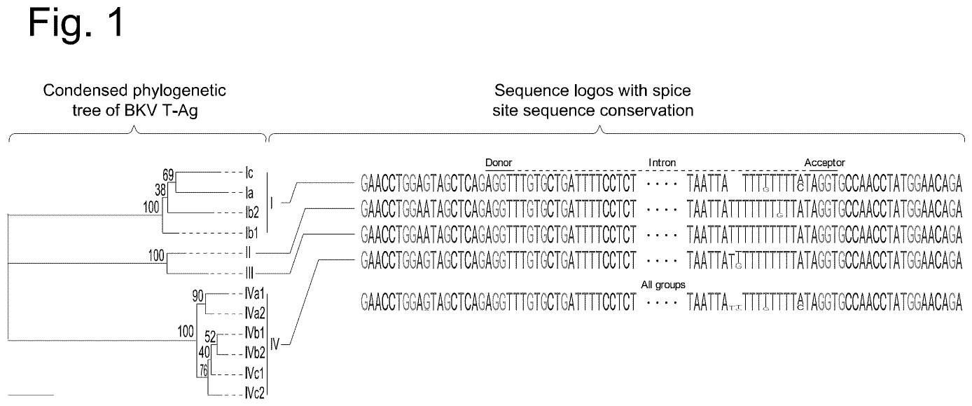

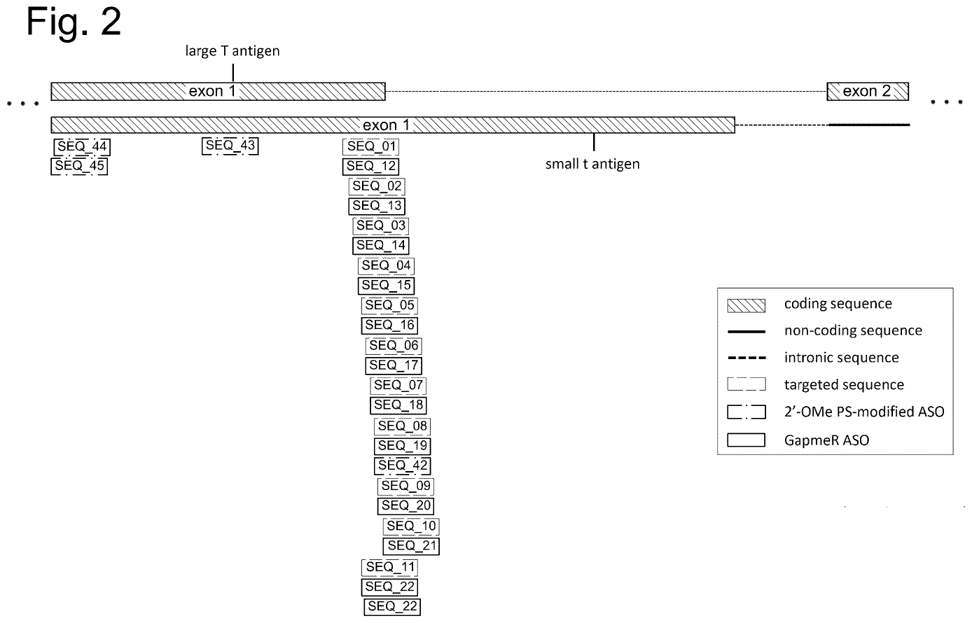

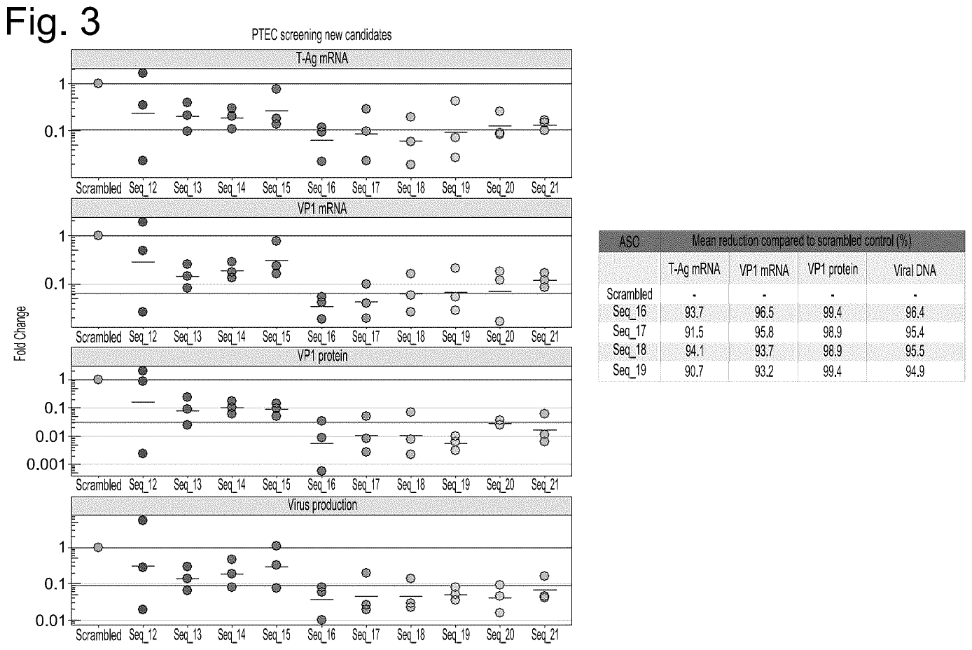

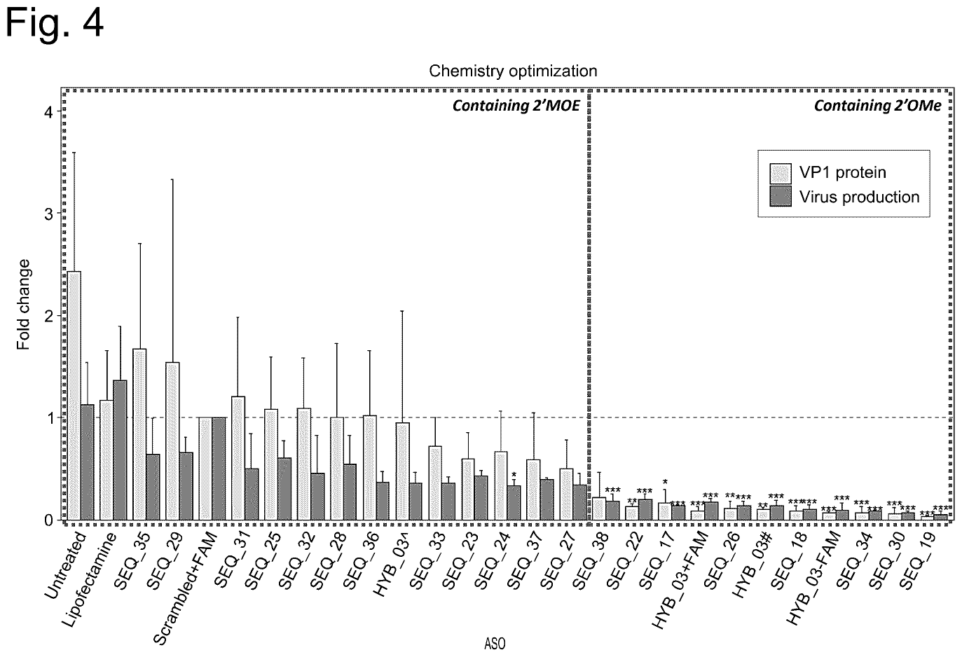

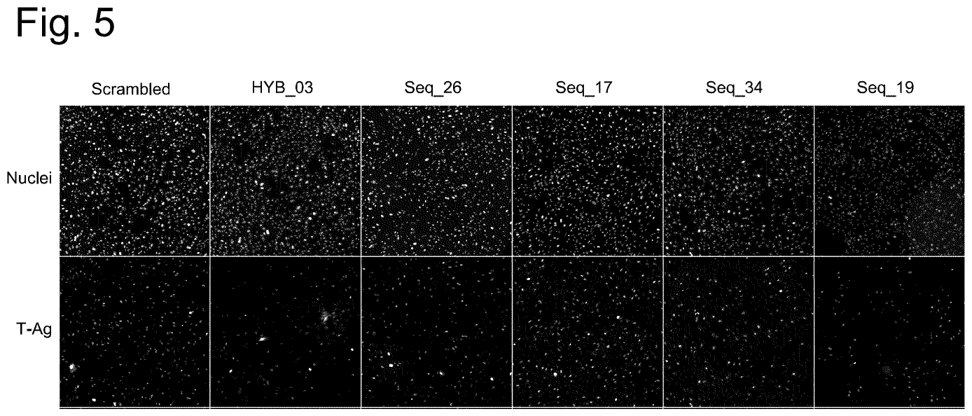

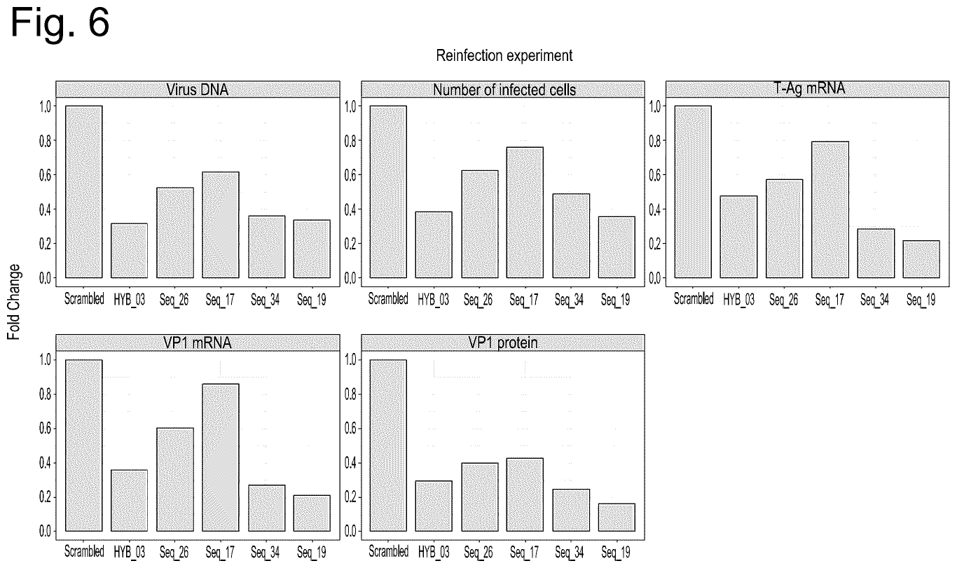

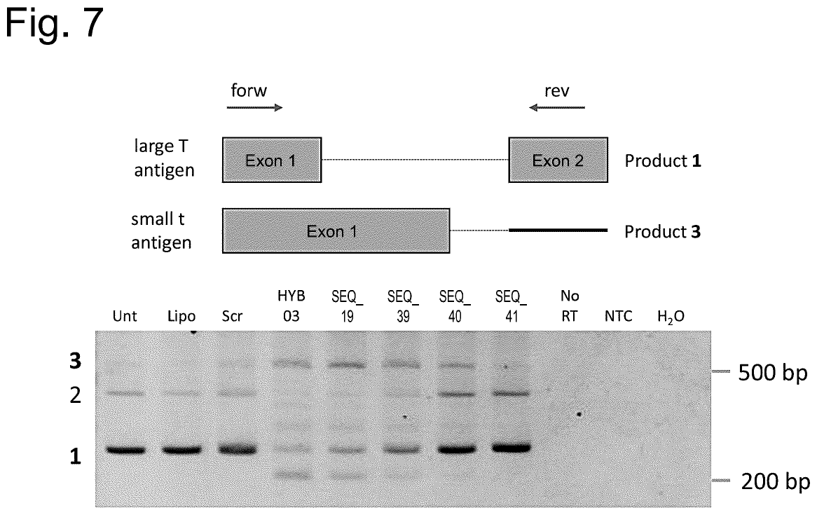

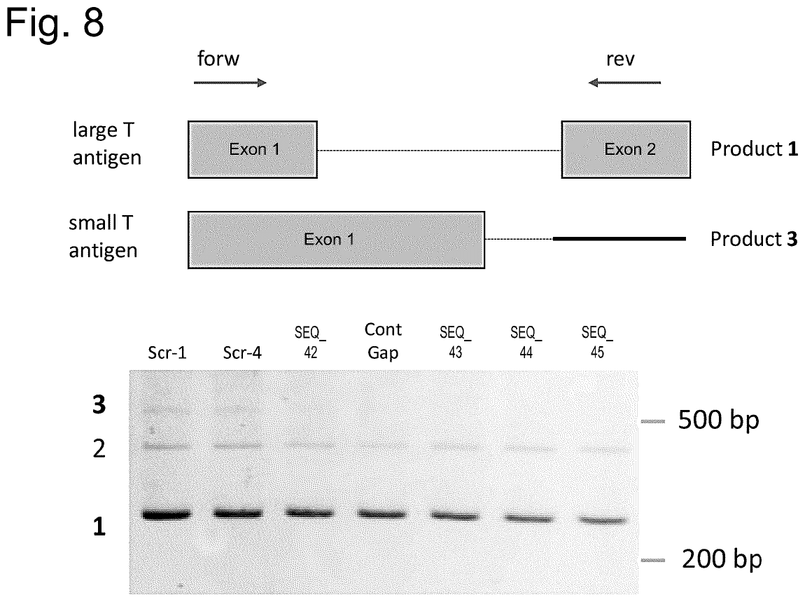

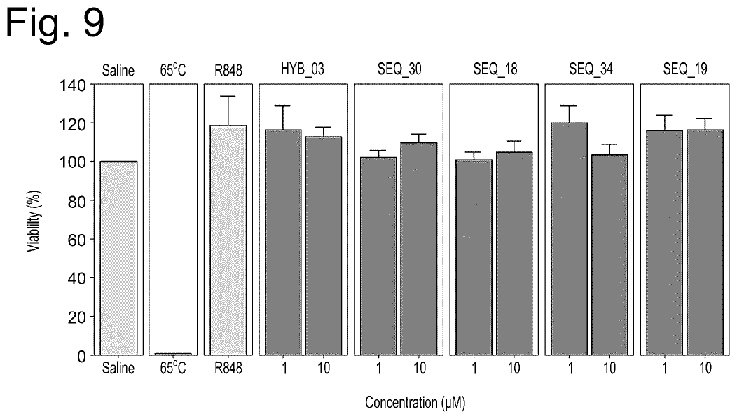

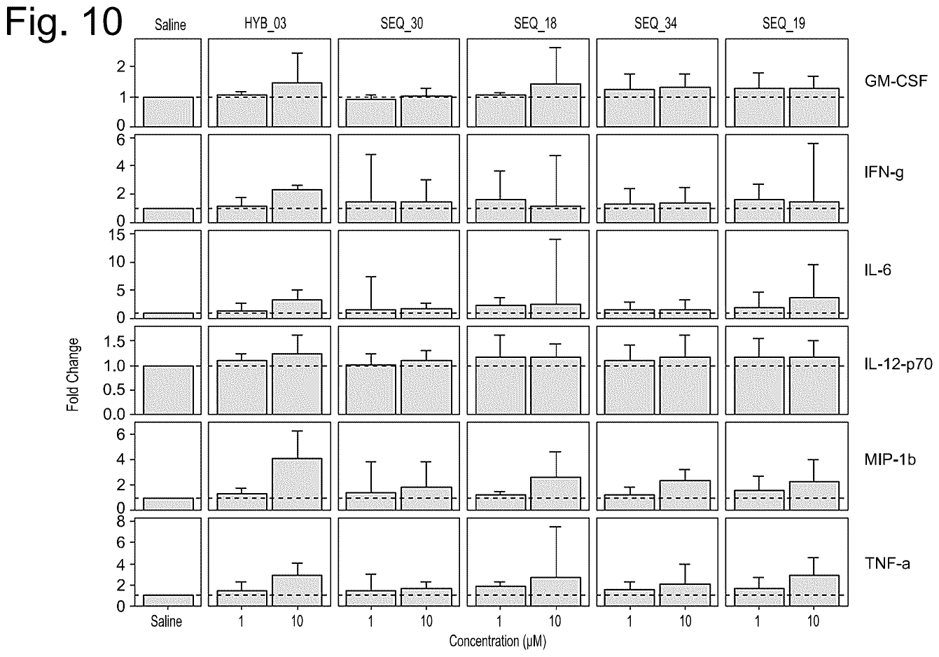

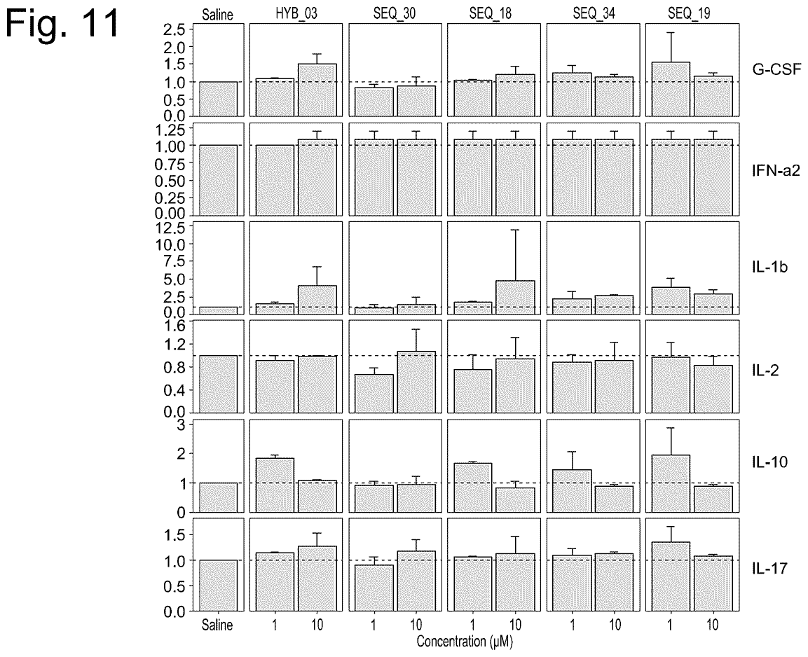

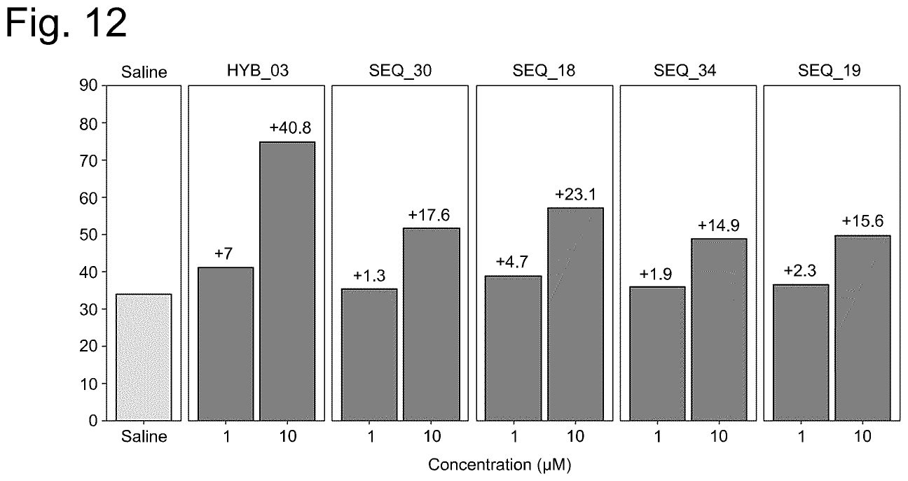

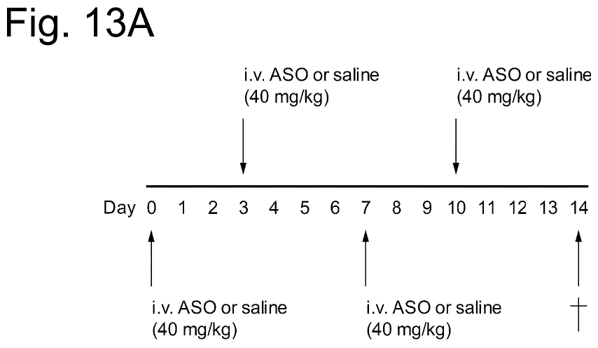

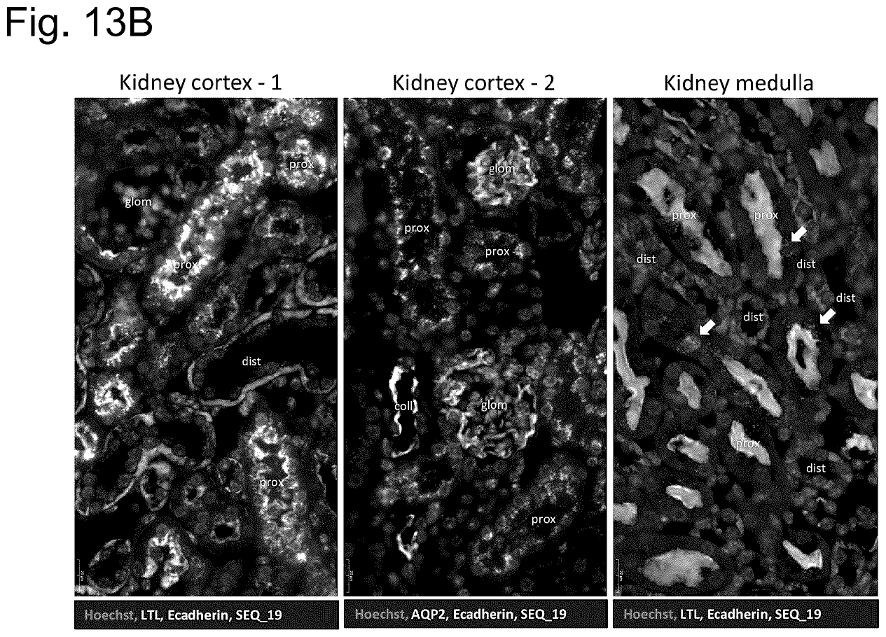

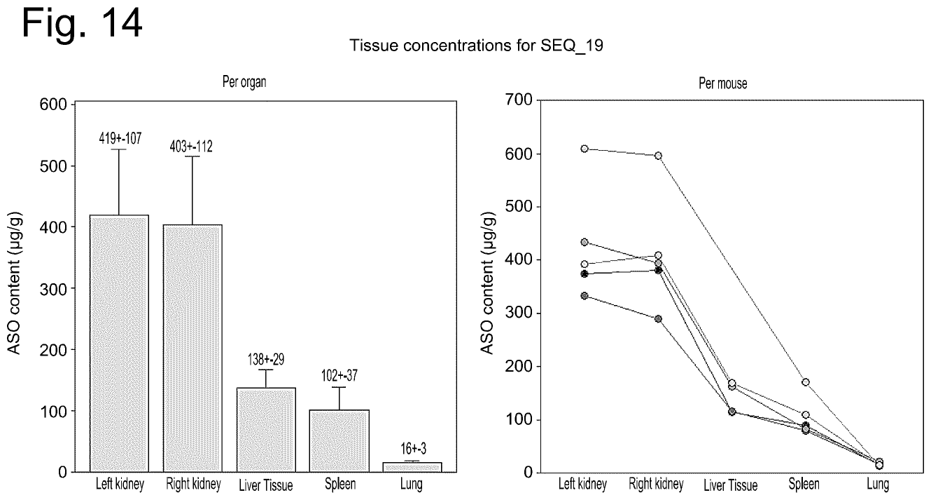

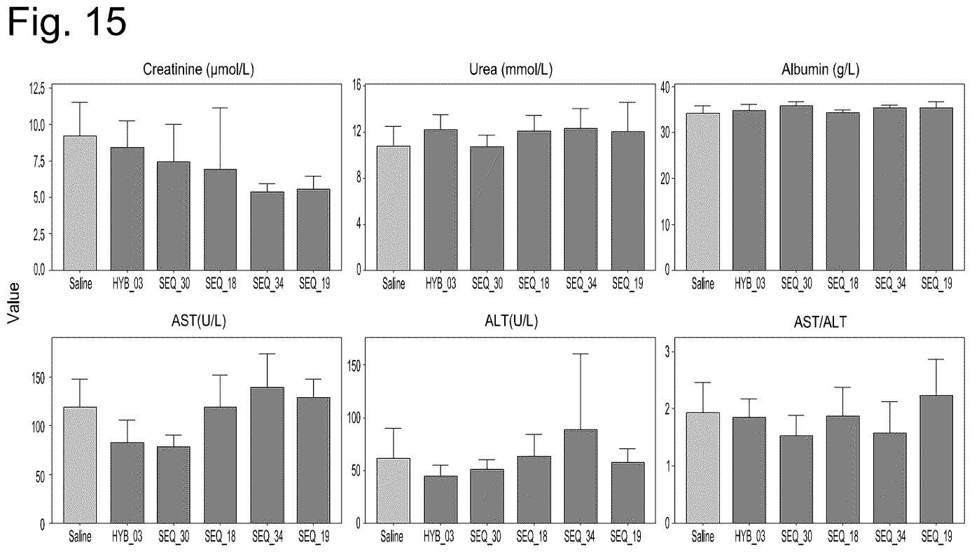

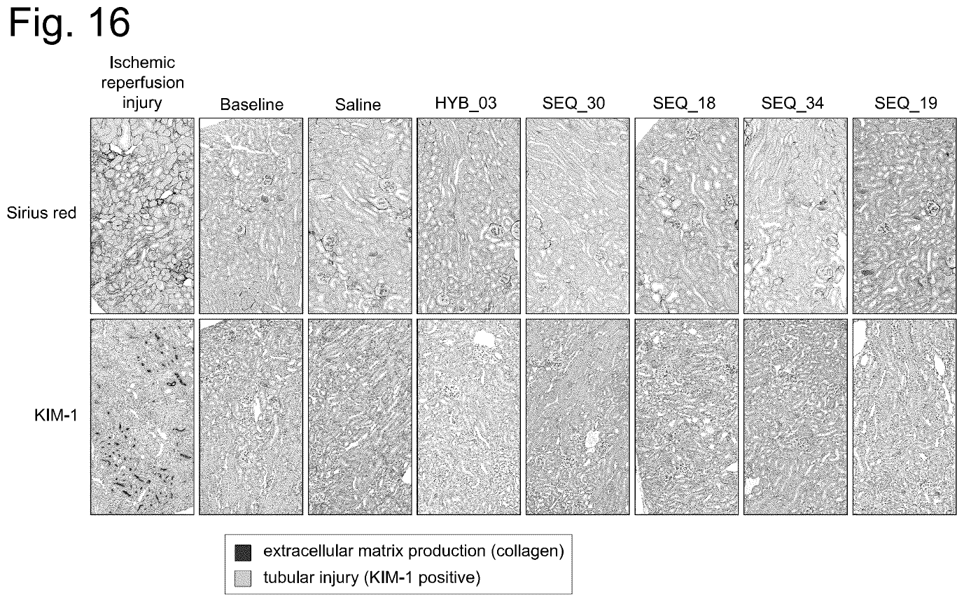

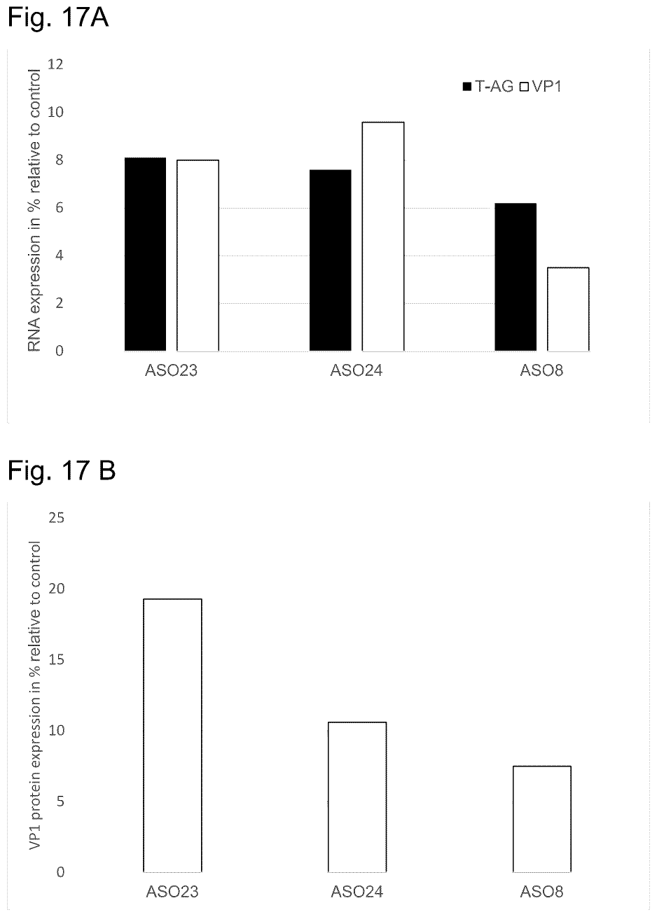

| SEQ | | |
|---|---|---|
| ID NO | Description | Sequences |
| 55 | Donor site I, IV | GAACTGGAGTAGCTCAGAGGTTTGTGCTGATTTTCCTCT |
| 56 | Donor site II, III | GAACTGGAATAGCTCAGAGGTTTGTGCTGATTTTCCTCT |
| 57 | Donor site consensus | GAACTGGARTAGCTCAGAGGTTTGTGCTGATTTTCCTCT |
| 58 | Acceptor site I | TAATTATTTKTTTTMTAGGTGCCAACCTATGGAACAGA |
| 59 | Acceptor site II | TAATTATTTTTTTKTTATAGGTGCCAACCTATGGAACAGA |
| 60 | Acceptor site III | TAATTATTTTTTTTTTATAGGTGCCAACCTATGGAACAGA |
| 61 | Acceptor site IV, without | TAATTATKTTTTTTTTATAGGTGCCAACCTATGGAACAGA |
| | gap | |
| 62 | Acceptor site IV, with gap | TAATTAKTTTTTTTTATAGGTGCCAACCTATGGAACAGA |
| 63 | Acceptor site consensus, | TAATTATKTTTKTTTTMTAGGTGCCAACCTATGGAACAGA |
| | without gaps | |
| 64 | Acceptor site consensus, | TAATTATTTTKTTTTMTAGGTGCCAACCTATGGAACAGA |
| | with 1 gap | |
| 65 | Acceptor site consensus, | TAATTAKTTTKTTTTMTAGGTGCCAACCTATGGAACAGA |
| | with 1 gap | |
| 66 | Acceptor site consensus, | TAATTATTTKTTTTMTAGGTGCCAACCTATGGAACAGA |
| | with 2 gaps | |

FIG. 2: Multiple ASOs were designed to target the exon 1—intronic donor site of BK virus large T antigen. Large T antigen donor splice sites being targeted are defined as SEQ ID NOs: 1 to 10 and were designed using a design algorithm developed in-house. In short, the algorithm considers antisense oligonucleotide (ASO) GO content (40-60%), melting temperature (>48° C.) and the absence of CpG motifs. A score is assigned to each potential ASO that increases when a requirement is not met. In general, ASOs with a low score are preferred over ASOs with a high score but predicted secondary structures of the target RNA and ASO are also considered, as well as the binding to splicing (RESCUE) enhancers and/or silencers in the target RNA. Importantly, the score considers whether design requirements are met, but it is not predictive for efficacy. Lastly, targetable sequences were required to contain at least 1 exonic or intronic nucleotide. The light boxes define 2′-OMe- fully modified ASOs (SEQ ID NOs: 12 to 21) derived from each of SEQ ID NOs: 1 to 10 and dark boxes define GapmeR oligonucleotides targeting either the exon—intron junction (SEQ ID NO: 42) or exon 1 sequence SEQ ID NOs: 43 to 45. A truncated ASO is represented by SEQ ID NO: 11. The ASO represented by SEQ ID NO: 11 comprises part of SEQ ID NO: 5. The ASO represented by SEQ ID NO: 11 has been further modified as a 2′-OMe fully-modified ASO (all nucleotides have a 2′-O-methyl base) and each internucleotide linkage is a phosphorothioate linkage. This further modified ASO is represented by SEQ ID NO: 22).

FIG. 3: Chemically-modified ASOs targeting BKV large T antigen exon 1—intron junction mediate 10- to 100-fold suppression of BKV parameters. Human primary kidney tubular epithelial cells (PTEC) were treated with new candidate 2′-OMe-modified ASOs (SEQ ID NOs: 12 to 21) and subsequently infected with BKV. BKV mRNA and protein expression and viral particle release into the supernatant were studied using PCR or western blot (n=3 replicates). Plots show fold changes relative to scrambled control for every individual ASO on a variety of outcome measures: T-Ag mRNA, VP1 mRNA, VP1 protein and virus production. The thick, dark grey line indicates mean overall reductions observed for HYB_03 (20-mer, 2′O-methyl nucleotides and phosphorothioate backbone, represented by SEQ ID NO: 68), which serves as a reference. SEQ ID NO: 67 is the native RNA sequence SEQ ID NO: 68 is derived from. SEQ ID NO: 67 is represented by 5′-CAGCACAAACCUCUGAGCUA-3′.

The variability in SEQ ID NO: 12 activity resulted from poor nuclear localization and viability of cells for the different replicates. A selection of new candidate sequences (SEQ ID NOs: 16 to 19) stood out due to their high potency to suppress BKV mRNA and protein expression, as well as virus production. In general, mRNA expression and production of virus particles were suppressed at least 10-fold, while protein expression was reduced 100-fold following treatment with these new 2′-OMe-modified ASO candidates, compared to the 2′-OMe-modified scrambled control.

FIG. 4: 2′-OMe chemistry yields greater reductions in BKV parameters than ASOs possessing 2′-MOE chemistry upon targeting the exon 1—intron junction. Human primary kidney tubular epithelial cells (PTEC) were treated with chemistry variations of new candidate ASOs (SEQ ID NOs: 16 to 19) and subsequently infected with BKV. BKV protein expression and viral particle release into the supernatant were studied using PCR or western blot. FAM-labeled scrambled control, HYB_03 with a FAM conjugate (i.e. SEQ ID NO: 68(+FAM)), as well as unlabeled HYB_03(-FAM) (i.e. SEQ ID NO: 68(-FAM)) were taken along as reference or control (n=3 replicates). Plots show fold changes relative to the FAM-labeled scrambled control for every individual ASO on a variety of outcome measures: VP1 protein and virus production. Interestingly, ASOs containing MOE-PS (SEQ ID NOs: 23, 24, 25, 27, 28, 29, 31, 32, 33, 35, 36, 37, 38, or 69), displayed considerably less activity in suppressing BKV while OMe-PS variations (SEQ ID NOs: 17, 18, 19, 22, 26, 30, 34, 38, and 68, HYB_03) showed strong suppression of protein expression and virus production. The presence of 5′-methylcytidines did not appear to interfere with ASO efficacy. The 4 most effective ASOs besides HYB_03 (SEQ ID NO: 68) were chosen as most promising candidates, being SEQ ID NOs: 30, 18, 34 and 19.

FIG. 5: The oligonucleotide with SEQ ID NO: 19 yields potent reductions of BKV infected cells following ‘reinfection’. Reinfection assays were performed to determine whether the ASOs limit the generation of active virus, whereby human proximal tubule epithelial cells were treated with either a scramble control and several ASOs that displayed activity against BKV (HYB_03 (SEQ ID NO: 68), SEQ ID NO: 26, SEQ ID NO: 17, SEQ ID NO: 34, SEQ ID NO: 19). Human PTECs were pre-treated with ASO (24 hours prior to infection) and infected with BKV, after which the cells were incubated for a period of 7 days. Culture supernatant was harvested on day 7 and placed in 10× diluted form on freshly plated PTECs for 3 days. Cells were stained for expression of nuclei (DAPI) and large T antigen protein.

FIG. 6: The oligonucleotide with SEQ ID NO: 19 yields potent reductions of BKV-derived RNA, protein and viral DNA following ‘reinfection’. Reinfection assays were performed to determine whether the ASOs limit the generation of active virus, whereby human proximal tubule epithelial cells were treated with either a scramble control and several ASOs that displayed activity against BKV (HYB_03 (SEQ ID NO: 68), SEQ ID NO: 26, SEQ ID NO: 17, SEQ ID NO: 34, SEQ ID NO: 19). Human PTECs were pre-treated with ASO (24 hours prior to infection) and infected with BKV, after which the cells were incubated for a period of 7 days. Culture supernatant was harvested on day 7 and placed in 10× diluted form on freshly plated PTECs for 3 days, at which point we assessed the levels of large T mRNA, VP1 mRNA and protein and viral DNA production.

FIG. 7: Splice-targeting ASOs induce aberrant splicing of the early coding region pre-mRNA alongside a shift from large T to small t antigen. Mouse Balb/c cells were transformed with the pRPc vector containing the early coding region of the Gardner BK virus strain (Negrini, M. et al., Cancer Research, 1992). This drives constitutive expression of the early coding region of BKV in these cells, as evidenced by abundant expression of the large T (product 1) and small t antigen mRNAs (product 3). Scramble control, HYB_03 (SEQ ID NO: 68) and SEQ ID NO: 19 were administered by lipofectamine at a concentration of 25 nM and RNA harvested 24 hours post-treatment. Mismatch controls of SEQ ID NO: 19 (single mismatch at nucleobase 10 (SEQ ID NO: 39), double mismatch at nucleobase 5 and 10 (SEQ ID NO:40) and triple mismatch at nucleobases 5, 10 and 15 (SEQ ID NO: 41)) were administered by lipofectamine at a concentration of 25 nM and RNA harvested 24 h after treatment. Note: These cells were kindly provided by Prof. Massimo Negrini (University of Ferrara, Italy). Treatment with HYB_03 and SEQ ID NO: 19 dramatically impacts splicing and expression levels of the early coding region pre-mRNA, resulting in a clear shift from large T antigen as the major product (band 1) to various RNA products, including an increase in small t antigen (band 3). Introduction of a single mismatch leads to partial restoration of normal splicing, whereas 2-3 mismatches almost completely restore splicing of large T antigen as the primary RNA species. These data provide clear evidence of target engagement and specificity.

FIG. 8: Large T Antigen splice-targeting GapmeRs do not induce aberrant splicing and mediate less meaningful reductions in large T antigen mRNA. Mouse Balb/c cells were transformed with the pRPc vector containing the early coding region of the Gardner BK virus strain (Negrini, M. et al., Cancer Research, 1992). This drives constitutive expression of the early coding region of BKV in these cells, as evidenced by abundant expression of the large T (product 1) and small t antigen mRNAs (product 3). As opposed to antisense oligonucleotides employing steric hindrance to modulate splicing, GapmeRs were designed deriving from the sequence of SEQ ID NO: 19 and administered by lipofectamine at a concentration of 25 nM and RNA harvested 24 hours post-treatment. A GapmeR version of SEQ ID NO: 19 was administered by lipofectamine at a concentration of 25 nM and RNA harvested 24 h after treatment (SEQ ID NO: 42), but did not display any notable activity with respect to large T or small t antigen RNA expression levels. Additional GapmeRs targeting exon 1 of the early coding region (pre-)mRNA (SEQ ID NOs: 43 to 45) did not mediate marked reductions in early coding region mRNAs. Hence, GapmeRs did not impact T-Antigen splicing as much as previously observed with the splice site-targeting ASOs HYB_03 (SEQ ID NO: 68) and SEQ ID NO: 19. These findings indicate that, although GapmeRs are capable of degrading the target mRNA, splicing of the target mRNA is largely unaffected. Note: These cells were kindly provided by Prof. Massimo Negrini (University of Ferrara, Italy).

FIG. 9: PBMC viability is not affected by treatment with SEQ ID NO: 30, SEQ ID NO: 18, SEQ ID NO: 34 and SEQ ID NO: 19. Healthy human peripheral blood mononuclear cells (PBMC) were isolated from buffy coats of 4 different donors and incubated for 48 hours in the presence of increasing concentrations of ASO. After incubation, cell viability was measured using the Cell titer Blue viability assay. PBMC were incubated for 10 minutes at 65° C. to serve as positive control. As an additional control, PBMC were activated using 1 μM R848 (TLR7/8 agonist) and all conditions were compared to saline-treated controls. Cell viability is expressed as a percentage relative to saline-treated control. As depicted in the figure, no viable cells were detected in the 65° C.-incubated positive control, while no significant effects on cell viability were observed in ASO-treated PBMC after 48 hours.

FIG. 10: Treatment with SEQ ID NO: 30, SEQ ID NO: 18, SEQ ID NO: 34 and SEQ ID NO: 19 mediates minimal activation of pro-inflammatory cytokine responses in PBMCs. Healthy human peripheral blood mononuclear cells (PBMC) were isolated from buffy coats of 4 different donors and incubated for 48 hours in the presence of increasing concentrations of ASO. After incubation, supernatant was harvested to measure cytokine production. As a positive control, PBMC were activated using 1 μM R848 (TLR7/8 agonist, data not shown) and all conditions were compared to saline-treated controls. Relative production and release of 6 cytokines in response to exposure to SEQ ID NO: 30, SEQ ID NO: 18, SEQ ID NO: 34 and SEQ ID NO: 19 is shown in the figure to the left. Some mild transient increase in some cytokine levels is observed in response to ASO treatment, but never to the same extent as the R848-treated control. Data represent cytokines within detection limits in all 4 donors.

FIG. 11: Treatment with SEQ ID NO: 30, SEQ ID NO: 18, SEQ ID NO: 34 and SEQ ID NO: 19 treatment mediates minimal activation of pro- and anti-inflammatory cytokines in PBMCs. Healthy human peripheral blood mononuclear cells (PBMC) were isolated from buffy coats of 4 different donors and incubated for 48 hours in the presence of increasing concentrations of SEQ ID NO: 30, SEQ ID NO: 18, SEQ ID NO: 34 and SEQ ID NO: 19. After incubation, supernatant was harvested to measure cytokine production. As a positive control, PBMC were activated using 1 μM R848 (TLR7/8 agonist, data not shown) and all conditions were compared to saline-treated controls. Relative production and release of 6 cytokines in response to treatment with SEQ ID NO: 30, SEQ ID NO: 18, SEQ ID NO: 34 and SEQ ID NO: 19 is shown in the figure to the left. Some mild transient increase in some cytokine levels is observed in response to ASO treatment, but never to the same extent as the R848-treated control. Data represent cytokines within detection limits in 2 out of 4 donors.

FIG. 12: Exposure of human plasma to the oligonucleotide having SEQ ID NO: 19, SEQ ID NO 30, SEQ ID NO: 18 or SEQ ID NO: 34 minimally impacts plasma coagulation time. Increasing concentrations of ASO were added to normal human plasma, after which the intrinsic pathway of the coagulation cascade was activated using the activated partial thromboplastin time (aPTT) test. ASO conditions were compared to saline-treated control plasma. Activation of the intrinsic coagulation cascade in saline-treated control plasma resulted in a coagulation time of 33.8 seconds. Addition of SEQ ID NO: 30, SEQ ID NO: 18, SEQ ID NO: 34 and SEQ ID NO: 19 to plasma resulted in a dose-dependent elongation of coagulation time. Data represents n=1.

FIGS. 13A-13B: BKV-targeting ASOs are clearly taken up in proximal and distal tubules of the mouse kidney cortex. FIG. 13A) 9 weeks old male C57BL6 mice received intravenous injections of 40 mg/kg HYB_03 (SEQ ID NO: 68), SEQ ID NO: 30, SEQ ID NO: 18, SEQ ID NO: 34 or SEQ ID NO: 19 at days 0, 3, 7, 10. These mice were compared to saline-treated animals. Group sizes were 5 mice per group per time point. FIG. 13B) Visual evidence of high levels of SEQ ID NO: 19 distribution to the kidney, and in particular the proximal tubule epithelial cells as evidenced by staining using a specific Cy5-labelled FISH probe (SEQ ID NO: 19). Individual segments of the nephron are defined as glomerulus (glom), proximal tubule epithelial cells (prox) and distal tubule epithelial cells (dist), and collecting duct epithelium (“coll”) based on immunohistochemical staining for Nephrin (“glom”), LTL (“prox”), E-cadherin (“dist”) and Aquaporin-2 (“coll”). B) (right image), trace levels of SEQ ID NO: 19 are detected in the medullary portion of the kidney (see arrows).

FIG. 14: Hybridization ELISA analysis of SEQ ID NO: 19 uptake for kidney and high bloodflow organs. 9-week old male C57BL6 mice received intravenous injections of 40 mg/kg HYB_03 (SEQ ID NO: 68), SEQ ID NO: 30, SEQ ID NO: 18, SEQ ID NO: 34 or SEQ ID NO: 19 at days 0, 3, 7, 10 and were compared to saline-treated animals (n=5 mice per group). The results are shown for experiments carried out with SEQ ID NO: 19. Left panel: Quantitation of SEQ ID NO: 19 levels in both kidneys (left and right), liver, spleen and lung are shown, revealing approximately 2.5-times greater (relative) uptake in kidney as compared to liver per gram of tissue. Similarly, greater relative uptake in kidney is observed as compared to lung and spleen. Right panel: Individual mouse tissue levels are depicted, indicating consistent tissue distribution profiles in vivo.

FIG. 15: Multiple dosing of SEQ ID NO: 30, SEQ ID NO: 18, SEQ ID NO: 34 or SEQ ID NO: 19 over 2 weeks does not result in signs of kidney or liver damage in the serum of mice. 9-week old male C57BL6 mice received intravenous injections of 40 mg/kg HYB_03 (SEQ ID NO: 68), SEQ ID NO: 30, SEQ ID NO: 18, SEQ ID NO: 34 or SEQ ID NO: 19 at days 0, 3, 7, 10 and were compared to saline-treated animals. Serum levels of markers of kidney and liver function were studied at 14 days after the first administration (n=5 mice per group). Top panels: Serum levels of kidney (Creatinine, Urea and Albumin) and liver (AST and ALT) biomarkers indicating organ function and/or injury. While some minor differences are detected between some groups, all levels were well within normal ranges, and therefore no signs of kidney or liver damage were observed.

FIG. 16: Repeated dosing of SEQ ID NO: 30, SEQ ID NO: 18, SEQ ID NO: 34 or SEQ ID NO: 19 over 2 weeks does not result in signs of kidney damage. 9 weeks old male C57BL6 mice received intravenous injections of 40 mg/kg HYB_03 (SEQ ID NO: 68), SEQ ID NO: 30, SEQ ID NO: 18, SEQ ID NO: 34 or SEQ ID NO: 19 at days 0, 3, 7, 10 and were compared to saline-treated animals (n=5 mice per group). Representative images of Sirius red (top panels) and KIM-1 staining (lower panels) in kidneys upon sacrifice. It is clear from the above figures that administration of saline (0.9% NaCl) or ASO did not result in ASO-related signs of kidney damage. Images were compared to a positive control (ischemia-reperfusion injury; IRI). B. Quantification of Sirius red staining in kidneys at day 14. Three animals that had received ASO showed an increase in kidney collagen content (HYB_03 n=2, SEQ ID 34, n=1), but this cannot be clearly attributed to administration of an ASO, and may be due to a potential higher number of major blood vessels in the images used for quantification.

FIGS. 17A-17B: ASOs according to the invention have improved antiviral activity. PTEC were treated with ASOs 24 h prior to infection with BKV, and BKV RNA expression was quantified at day 5 post-infection. FIG. 17A) Relative T-Ag and VP1 RNA expression of cells treated with ASO8 (SEQ ID NO: 8), ASO23 (SEQ ID NO: 23 of WO2019/168402) and ASO24 (SEQ ID NO: 24 of WO2019/168402). FIG. 17B) Relative VP1 protein expression of cells treated with ASO8, ASO23 and ASO24.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturers' specifications, instructions etc.), whether supra or infra, is hereby incorporated by reference in its entirety.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments, which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the word “comprise”, and variations such as “comprises” and “comprising”, are to be understood to imply the inclusion of a stated integer or step or group of integers or steps but not the exclusion of any other integer or step or group of integer or step. In preferred embodiments, “comprise” can mean “consist of”. As used in this specification and the appended claims, the singular forms “a”, “an”, and “the” include plural referents, unless the content clearly dictates otherwise.

In a first aspect, the invention relates to an oligonucleotide, specifically to an oligonucleotide comprising a nucleobase sequence having the base-pairing specificity of a nucleobase sequence according to one of SEQ ID NOs: 1 to 11.

Typically, this oligonucleotide comprises a nucleobase sequence according to one of SEQ ID NOs: 1 to 11 or comprises a nucleobase sequence which is analogous to any one of SEQ ID NOs: 1 to 11 characterized in that at least one nucleobase of said SEQ ID NOs is replaced by a nucleobase analogue having the same base pairing specificity as the replaced nucleobase.

### Oligonucleotide, Basic Structure

An oligonucleotide of the invention is capable of specifically binding to a polyomavirus pre-mRNA produced upon polyomavirus infection of a human cell (“target RNA”). As such, it can also be described as a polyomavirus oligonucleotide or antisense oligonucleotide (“ASO”) or polyomavirus antisense oligonucleotide. The part (i.e. stretch of contiguous nucleobases) of the target RNA the oligonucleotide is capable of specifically binding to is referred to herein as the “target region”.

In a preferred embodiment, at least a part of the oligonucleotide is at least substantially complementary to the target region. As such, the oligonucleotide comprises at least 12 contiguous nucleobases with a sequence that is the reverse complement of the sequence of at least 12 contiguous nucleobases of the target region. Preferably, the target region is 12 to 30, i.e. 12, 13, 14, 15, 16, 17, 18, 19, 20, 21, 22, 23, 24, 25, 26, 27, 28, 29 or 30 nucleobases, or 12 to 28 nucleobases, in length. More preferably, the target region is 17 to 26, i.e. 17, 18, 19, 20, 21, 22, 23, 24, 25 or 26 nucleobases in length. Even more preferably, the target region is 18 to 22, i.e. 18, 19, 20, 21 or 22 nucleobases in length. Most preferably, the target region is 20 nucleobases in length.

Generally, the target region comprises polyomavirus pre-RNA. The target region preferably comprises the splice donor site of intron 1 (or part thereof) and/or exon 1 (or part thereof) of the large T-antigen of the respective polyomavirus. Preferably, the target region comprises up to 30 contiguous nucleobases of intron 1 and/or (preferably and) exon 1, wherein 0 to 30 of the 30 contiguous nucleobases, i.e. 0, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 contiguous nucleobases are of the splice donor site of intron 1 and the remaining contiguous nucleobases are of exon 1. In a preferred embodiment, the target region comprises one or two intron nucleobases adjacent to the splice donor site. In a preferred embodiment the target region comprises 1-28, i.e. 1, 2, 3, 4, 5, 6, 7, 8, 9, 10, 11, 12, 13, 14, 15, 16, 17, 18, 19, 20, 21, 22, 23, 24, 25, 26, 27 or 28 intron nucleobases adjacent to the splice donor site. In another preferred embodiment, the target region comprises 1-28, i.e. 1, 2, 3, 4, 5, 6, 7, 8, 9, 10, 11, 12, 13, 14, 15, 16, 17, 18, 19, 20, 21, 22, 23, 24, 25, 26, 27 or 28 exon nucleobases adjacent to the splice donor site.

It is to be understood that the sum of the number of intron and exon nucleobases does not exceed the total number of nucleobases in the oligonucleotide.

In an embodiment, the oligonucleotide may comprise nucleobase(s) that is (are) not capable of specifically binding to the target RNA, and is (are) in particular not reverse complementary to the target RNA in the context of the contiguous sequence that is reverse complementary to the target RNA.

The length of the oligonucleotide is up to 200, up to 175 or up to 150, preferably up to 100 and more preferably up to 50 nucleobases (including nucleotides and nucleotide analogues) long, e.g. 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleobases. When the length of the oligonucleotide is greater than 50 nucleotides (for example when the length is 75 or 100 or 150 or 200 nucleotides), said oligonucleotides may alternatively be called a polynucleotide. In preferred embodiments, the oligonucleotide has a length of 12 to 27, preferably 12 to 22, i.e. 12, 13, 14, 15, 16, 17, 18, 19, 20, 21, or 22 nucleobases, more preferably from 17, 18, 19 or 20 to 22 nucleobases, i.e. 17, 18, 19, 20, 21 or 22 nucleotides. In a preferred embodiment, the oligonucleotide has a length of 20 nucleobases.

Specifically, the first aspect relates to an oligonucleotide comprising a nucleobase sequence (or the nucleotide sequence) having the base-pairing specificity of a nucleobase sequence according to one of SEQ ID NOs: 1 to 11. This means that the nucleobase sequence of the oligonucleotide comprises the nucleobase sequence according to one of SEQ ID NOs: 1 to 11, or it comprises a nucleobase sequence that is analogous to any one of SEQ ID NOs: 1 to 11. “Analogous” means that it is characterized in that at least one nucleobase of said SEQ ID NOs is replaced by a nucleobase analogue having the same base pairing specificity as the replaced nucleobase. Nucleobase analogues are modifications explained below. Preferred embodiments relate to SEQ ID NOs: 5, 6, 7, 8 and 10, preferably SEQ ID NO: 8. Embodiments of the oligonucleotide described above, in as far as compatible, apply. In a preferred embodiment, the nucleobase sequence (or the nucleotide sequence) of the oligonucleotide consists of the nucleobase sequence according to one of SEQ ID NOs: 1 to 11, or wherein the nucleobase sequence of the oligonucleotide consists of a nucleobase sequence that is analogous to any one of SEQ ID NOs: 1 to 11.

### Oligonucleotide, Modifications

The oligonucleotide may be modified (referred to herein as “modified oligonucleotide”). It is expected that this improves stability, in particular resistance to nucleases. This is an advantage when the oligonucleotide is administered as such (i.e. naked administration) to a patient.

Accordingly, in an embodiment, the oligonucleotide is a modified oligonucleotide. In an embodiment, the modification of said oligonucleotide is compared to a (natural) RNA oligonucleotide. A modified oligonucleotide comprises a modified internucleotide linkage and/or a modified nucleotide. A modified nucleotide is synonymous of nucleotide analogue.

In a preferred embodiment, the oligonucleotide comprises a modification capable of rendering an RNA duplex resistant to nucleases (preferably exonucleases, in particular RNase H), wherein the RNA duplex comprises the oligonucleotide and an oligonucleotide at least partially complementary thereto (complementary oligonucleotide, i.e. the target RNA). In other words, the oligonucleotide provides resistance to degradation by nucleases of a RNA duplex comprises the oligonucleotide and an oligonucleotide at least partially complementary thereto. This is preferably afforded by internucleotide linkage modifications (e.g. phosphorothioate modified nucleotides and/or sugar (scaffold) modifications (e.g. 2′-O-modifications sugar modifications) as described above. The oligonucleotide thus preferably comprises a region (i.e. at least 2, 3, 4, 5, 6, 7, 8, 9, or 10 modified nucleotides) with modifications that provide nuclease resistance to the duplex.

The modified oligonucleotide may comprise a nucleotide analogue and/or a modified internucleotide linkage. When each of the internucleotide linkage is modified, the oligonucleotide is said to have a “backbone modification”.

A nucleotide analogue preferably is a nucleotide that comprises a base modification (“modified base”) and/or a sugar modification (“modified sugar”, also referred to as scaffold modification). In an embodiment, a base modification is a modified version of the natural purine and pyrimidine bases (e.g. adenine, uracil, guanine, cytosine, and thymine), such as hypoxanthine, pseudouracil, pseudocytosine, 1-methylpseudouracil, orotic acid, agmatine, lysidine, 2-thiopyrimidine (e.g. 2-thiouracil, 2-thiothymine), G-clamp and its derivatives, 5-substituted pyrimidine (e.g. 5-halouracil, 5-halomethyluracil, 5-trifluoromethyluracil, 5-propynyluracil, 5-propynylcytosine, 5-aminomethyluracil, 5-hydroxymethyluracil, 5-aminomethylcytosine, 5′-methylcytosine, 5′-methylcytidine, 5-hydroxymethylcytosine, Super T, or as described in e.g. Kumar et al. J. Org. Chem. 2014, 79, 5047; Leszczynska et al. Org. Biol. Chem. 2014, 12, 1052), pyrazolo[1,5-a]-1,3,5-triazine C-nucleoside (as in e.g. Lefoix et al. J. Org. Chem. 2014, 79, 3221), 7-deazaguanine, 7-deazaadenine, 7-aza-2,6-diaminopurine, 8-aza-7-deazaguanine, 8-aza-7-deazaadenine, 8-aza-7-deaza-2,6-diaminopurine, Super G, Super A, boronated cytosine (as in e.g. Niziol et al. Bioorg. Med. Chem. 2014, 22, 3906), pseudoisocytidine, C(Pyc) (as in e.g. Yamada et al. Org. Biomol. Chem. 2014, 12, 2255) and N4-ethylcytosine, or derivatives thereof; N²-cyclopentylguanine (cPent-G), N²-cyclopentyl-2-aminopurine (cPent-AP), and N²-propyl-2-aminopurine (Pr-AP), carbohydrate-modified uracil (as in e.g. Kaura et al. Org. Lett. 2014, 16, 3308), amino acid modified uracil (as in e.g. Guenther et al. Chem. Commun. 2014, 50, 9007); or derivatives thereof; or a degenerate or universal base, like 2,6-difluorotoluene, or an absent bases like abasic sites (e.g. 1-deoxyribose, 1,2-dideoxyribose, 1-deoxy-2-O-methylribose; or pyrrolidine derivatives in which the ring oxygen has been replaced with nitrogen (azaribose)). Examples of derivatives of Super A, Super G and Super T can be found in U.S. Pat. No. 6,683,173 (Epoch Biosciences). cPent-G, cPent-AP and Pr-AP were shown to reduce immunostimulatory effects when incorporated in siRNA (Peacock H. et al. J. Am. Chem. Soc. 2011, 133, 9200). Further examples of modified bases are described in e.g. WO2014/093924.

A preferred modified base is 5′-methylcytosine and 5′-methylcytidine. In a preferred embodiment, all cytosines of the oligonucleotide are modified as (i.e. replaced by) 5′-methylcytosine or, preferably, 5′-methylcytidine.

Generally, a nucleobase analogue is used, at least in the part of the oligonucleotide which is complementary to the target region, to replace a nucleobase with the same base pairing specificity. “Base pairing” refers to the binding of two nucleobases to each other by hydrogen bonds. Specifically, a nucleobase analogue replacing cytosine is capable of base pairing with guanine, a nucleobase analogue replacing guanine is capable of base pairing with cytosine, a nucleobase analogue replacing adenine is capable of base pairing with uracil, and a nucleobase analogue replacing uracil is capable of base pairing with adenine.

The oligonucleotide may comprise at least 1, e.g. at least 1, 2, 3, 4, 5, 6, 7, 8, 9, or at least 10 base modifications. These may all be the same type of modification, or they may comprise two or more different modifications.

A sugar (scaffold) modification may be a modification of the ribosyl moiety, e.g. a 2′-O-modified RNA nucleotide such as 2′-O-alkyl or 2′-O-(substituted)alkyl e.g. 2′-O-methyl, 2′-O-(2-cyanoethyl), 2′-O-(2-methoxy)ethyl (2′-MOE), 2′-O-(2-thiomethyl)ethyl, 2′-O-butyryl, 2′-O-propargyl, 2′-O-acetalester (such as e.g. Biscans et al. Bioorg. Med. Chem. 2015, 23, 5360), 2′-O-allyl, 2′-O-(2S-methoxypropyl), 2′-O—(N-(aminoethyl)carbamoyl)methyl) (2′-AECM), 2′-O-(2-carboxyethyl) and carbamoyl derivatives (Yamada et al. Org. Biomol. Chem. 2014, 12, 6457), 2′-O-(2-amino)propyl, 2′-O-(2-(dimethylamino)propyl), 2′-O-(2-amino)ethyl, 2′-O-(2-(dimethylamino)ethyl); 2′-deoxy (DNA); 2′-O-(haloalkoxy)methyl (Arai K. et al. Bioorg. Med. Chem. 2011, 21, 6285) e.g. 2′-O-(2-chloroethoxy)methyl (MCEM), 2′-O-(2,2-dichloroethoxy)methyl (DCEM); 2′-O-alkoxycarbonyl e.g. 2′-O-[2-(methoxycarbonyl)ethyl](MOCE), 2′-O-[2-(N-methylcarbamoyl)ethyl](MCE), 2′-O-[2-(N,N-dimethylcarbamoyl)ethyl](DCME), 2′-O-[2-(methylthio)ethyl](2′-MTE), 2′-(w-O-serinol); 2′-halo e.g. 2′-F, FANA (2′-F arabinosyl nucleic acid); 2′,4′-difluoro-2′-deoxy; carbasugar and azasugar modifications; other modified RNA nucleotides are 3′-O-substituted (e.g. 3′-O-methyl, 3′-O-butyryl, 3′-O-propargyl), 4′-substituted (e.g. 4′-aminomethyl-2′-O-methyl or 4′-aminomethyl-2′-fluoro; 5′-substituted e.g. 5′-methyl), or CNA (Ostergaard et al. ACS Chem. Biol. 2014, 22, 6227). Derivatives of the foregoing are also envisaged.

Furthermore, a sugar (scaffold) modification can include a bicyclic nucleic acid monomer (BNA) which may be a bridged nucleic acid monomer. Each occurrence of said BNA may result in a monomer that is independently chosen from the group consisting of a conformationally restricted nucleotide (CRN) monomer, a locked nucleic acid (LNA) monomer, a xylo-LNA monomer, an α-LNA monomer, an α-L-LNA monomer, a β-D-LNA monomer, a 2′-amino-LNA monomer, a 2′-(alkylamino)-LNA monomer, a 2′-(acylamino)-LNA monomer, a 2′-N-substituted-2′-amino-LNA monomer, a 2′-thio-LNA monomer, a (2′-0,4′-C) constrained ethyl (cEt) BNA monomer, a (2′-0,4′-C) constrained methoxyethyl (cMOE) BNA monomer, a 2′,4′-BNA^{NC}(N—H) monomer, a 2′,4′-BNA^{NC}(N-Me) monomer, a 2′,4′-BNA^{NC}(N-Bn) monomer, an ethylene-bridged nucleic acid (ENA) monomer, a carba LNA (cLNA) monomer, a 3,4-dihydro-2H-pyran nucleic acid (DpNA) monomer, a 2′-C-bridged bicyclic nucleotide (CBBN) monomer, a heterocyclic-bridged BNA monomer (such as triazolyl or tetrazolyl-linked), an amido-bridged BNA monomer, an urea-bridged BNA monomer, a sulfonamide-bridged BNA monomer, a bicyclic carbocyclic nucleotide monomer, a TriNA monomer, an α-L-TriNA monomer, a bicyclo DNA (bcDNA) monomer, an F-bcDNA monomer, a tricyclo DNA (tcDNA) monomer, an F-tcDNA monomer, an oxetane nucleotide monomer, a locked PMO monomer derived from 2′-amino-LNA, a guanidine-bridged nucleic acid (GuNA) monomer, a spirocyclopropylene-bridged nucleic acid (scpBNA) monomer, and derivatives thereof.

A preferred sugar modification is selected from the group consisting of a 2′-O-modification, preferably 2′-O-alkyl or 2′-O-(substituted)alkyl, more preferably 2′-O-methyl or 2′-O-(2-methoxy)ethyl (2′-MOE), and a modification to a BNA monomer, preferably a CRN monomer or a locked nucleic acid (LNA) monomer. More preferred is the combination of a 2′-O-methyl sugar modification and a modification to an LNA. Even more preferred is 2′-O-methyl as the only sugar modification.

The oligonucleotide may comprise at least 1, e.g. at least 1, 2, 3, 4, 5, 6, 7, 8, 9, or at least 10 sugar modifications. These may all be the same type of modification, or they may comprise two or more different modifications.

An internucleotide linkage modification can be selected from the group consisting of: a modified phosphodiester of RNA, such as phosphorothioate (PS), chirally pure phosphorothioate, (R)-phosphorothioate, (S)-phosphorothioate, phosphorodithioate (PS2), phosphonoacetate (PACE), phosphonoacetamide (PACA), thiophosphonoacetate (thioPACE), thiophosphonoacetamide, phosphorothioate prodrug, H-phosphonate, methyl phosphonate, methyl phosphonothioate, methyl phosphate, methyl phosphorothioate, ethyl phosphate, ethyl phosphorothioate, boranophosphate, boranophosphorothioate, methyl boranophosphate, methyl boranophosphorothioate, methyl boranophosphonate, methyl boranophosphonothioate, phosphate, phosphotriester, aminoalkylphosphotriester, and their derivatives. Another modification includes phosphoryl guanidine, phosphoramidite, phosphoramidate, N3′→P5′ phosphoramidate, phosphordiamidate, phosphorothioamidate, sulfamate, dimethylenesulfoxide, amide, sulfonate, siloxane, sulfide, sulfone, formacetyl, thioformacetyl, methylene formacetyl, alkenyl, methylenehydrazino, sulfonamide, amide, triazole, oxalyl, carbamate, methyleneimino (MMI), and thioacetamido nucleic acid (TANA); as well as their derivatives. Examples of chirally pure phosphorothioate linkages are described in e.g. WO2014/010250 or WO2017/062862 (WaVe Life Sciences). Examples of phosphoryl guanidine linkages are described in WO2016/028187 (Noogen). Various salts, mixed salts and free acid forms are also included, as well as 3′→3′ and 2′→5′ linkages.

The oligonucleotide may comprise at least 1, e.g. at least 1, 2, 3, 4, 5, 6, 7, 8, 9, or at least 10 internucleotide linkage modifications. These may all be the same type of modification, or they may comprise two or more different modifications.

A preferred internucleotide linkage modification is PS, PS2, phosphoramidate or phosphordiamidate, preferably PS. In a preferred embodiment, all internucleotide linkages of the oligonucleotide are PS linkages. In other words, the backbone of the oligonucleotide is PS.

In one embodiment, one of more nucleotides at the 5′ end of the oligonucleotide and/or one of more nucleotides at the 3′ end of the oligonucleotide are modified (i.e. are nucleotide analogs and/or have a modified internucleotide linkage), and nucleotides of the central part of the oligonucleotide are not modified. “Nucleotides at the 5′ or 3′ end” encompasses 20% of the nucleotides of the oligonucleotide at the respective end, and the “central part” encompasses the remaining nucleotides of the oligonucleotide. For example, if the oligonucleotide has a sequence consisting of 20 nucleotides, 4 nucleotides at the 5′ end of the oligonucleotide are nucleotides at the 5′ end, 4 nucleotides at the 3′ end of the oligonucleotide are nucleotides at the 3′ end, and the remaining 12 nucleotides are nucleotides of the central part of the oligonucleotide. If the number of nucleotides according to the percentages do not result in integers, it is rounded to the nearest integer (down for a first decimal of up to 4, and up for first decimal of at least 5). Accordingly, in one embodiment, at least one nucleotide and/or at least one internucleotide linkage that is present at the 5′ and/or at the 3′end of the oligonucleotide are modified, whereas other nucleotides and other internucleotide linkage are not modified.

Modifications encompassed have been all defined herein. It is expected that modifying the oligonucleotide at such places may contribute to improve its stability or resistance to exonucleases. This is an advantage when the oligonucleotide is administered as such to a patient (i.e. naked administration).

In an embodiment, the last 1, 2, 3, 4 nucleotides and/or internucleotide linkages at the 5′ of the oligonucleotide are modified.

In an embodiment, the last 1, 2, 3, 4 nucleotides and/or internucleotide linkages at the 3′ of the oligonucleotide are modified.

In an embodiment, the last 1, 2, 3, 4 nucleotides and/or internucleotide linkages at the 5′ and at the 3′ of the oligonucleotide are modified. Preferably, 2 nucleotides and/or 2 internucleotide linkages at the 5′ and at the 3′ of the oligonucleotide are modified. Preferably, 3 nucleotides and/or internucleotide linkages at the 5′ and at the 3′ of the oligonucleotide are modified. Preferably, 4 nucleotides and/or internucleotide linkages at the 5′ and at the 3′ of the oligonucleotide are modified.

For example, with regard to SEQ ID NOs: 1 to 10, the oligonucleotide comprises unmodified internucleotide linkages between nucleotides 5 to 16 of SEQ ID NOs: 1 to 10 and modified internucleotide linkages between at least the two most 5′ end nucleotides of the oligonucleotide and between at least the two most 3′ end nucleotides of the oligonucleotide. For example, with regard to SEQ ID NO: 11, the oligonucleotide comprises unmodified internucleotide linkages between nucleotides 5 to 15 of SEQ ID NO: 11 and modified internucleotide linkages between at least the two most 5′ end nucleotides of the oligonucleotide and between at least the two most 3′ end nucleotides of the oligonucleotide.

It is preferred that at least 1 and preferably 2 to 4, more preferably 4 nucleotides at the 5′ and/or at the 3′ end, and optionally all nucleotides of the oligonucleotide are modified, in particular by having a modified internucleotide linkage.

Modifications, in particular of nucleotides at the 5′ and/or 3′ end, preferably comprise a modified internucleotide linkage, more preferably to PS. In addition, the nucleotide preferably is a nucleotide analogue. This nucleotide analogue is characterized by comprising a modified sugar (preferably 2′-O-methyl), a modified base (preferably 5′-methylcytosine), and/or being an LNA monomer. For example, the following combinations characterizing the nucleotide analogue are envisaged: modified sugar only; modified sugar and modified base; LNA monomer only; LNA monomer and modified base.

In a preferred embodiment, all internucleotide linkage of the oligonucleotide are PS linkages and all nucleotides of the oligonucleotide have a 2′-O-methyl base. Therein, preferably all cytidines of the oligonucleotide are modified to 5-methylcytidines (i.e. the oligonucleotide comprises no cytidines, but 5-methylcytidines, in particular at positions paring with guanosine in the target RNA).

Accordingly, in an embodiment, when one refers to the modifications at the 5′ and/or 3′end of the oligonucleotide, such modifications are:
the modified internucleotide linkage is PS, and/or
the modified sugar is 2′-O-methyl
the modified base is 5-methylcytosine and/or
the modified nucleotide is a locked nucleic acid (LNA) monomer.

Accordingly, in an embodiment, when one refers to the modifications at the 5′ and/or 3′end of the oligonucleotide, such modifications are:
the modified internucleotide linkage is PS,
the modified sugar is 2′-O-methyl,
the modified base is 5-methylcytosine and/or
the modified nucleotide is a locked nucleic acid (LNA) monomer.

Preferably, such modifications are:
the modified internucleotide linkage is PS and
the modified sugar is 2′-O-methyl.

Preferably, such modifications are:
the modified internucleotide linkage is PS,
the modified sugar is 2′-O-methyl and
the modified base is 5-methylcytosine.

Preferably, such modifications are:
the modified internucleotide linkage is PS and
the modified nucleotide is a locked nucleic acid (LNA) monomer.

Preferably, such modifications are:
the modified internucleotide linkage is PS,
the modified nucleotide is a locked nucleic acid (LNA) monomer and
the modified base is 5-methylcytosine.

In an embodiment, the oligonucleotide is as follows: the internucleotide linkages of the central part of the oligonucleotide have not been modified and preferably the internucleotide linkages at the 2 to 4 most 5′-end and/or 2 to 4 most 3′-end of the oligonucleotide have been modified, preferably as phosphorothioate internucleotide linkage.

In an embodiment, the oligonucleotide has been modified as follows: each internucleotide linkage is a phosphorothioate linkage and all nucleotides have a 2′-O-methyl base.

In a preferred embodiment, the oligonucleotide has been modified as follows: each internucleotide linkage is a phosphorothioate linkage, all cytidine are 5′-methylcytidine and all nucleotides have a 2′-O-methyl base.

Examples of preferred oligonucleotides are characterized as follows:
Each internucleotide linkage is a phosphorothioate linkage and all nucleotides have a 2′-O-methyl base, e.g.: oligonucleotide with a nucleotide sequence comprising or consisting of the sequence of SEQ ID NO: 12 (nucleobase sequence of SEQ ID NO: 1), SEQ ID NO: 13 (nucleobase sequence of SEQ ID NO: 2), SEQ ID NO: 14 (nucleobase sequence of SEQ ID NO: 3), SEQ ID NO: 15 (nucleobase sequence of SEQ ID NO: 4), SEQ ID NO: 16 (nucleobase sequence of SEQ ID NO: 5), SEQ ID NO: 17 (nucleobase sequence of SEQ ID NO: 10), SEQ ID NO: 18 (nucleobase sequence of SEQ ID NO: 7), SEQ ID NO: 19 (nucleobase sequence of SEQ ID NO: 8), SEQ ID NO: 20 (nucleobase sequence of SEQ ID NO: 9), SEQ ID NO: 21 (nucleobase sequence of SEQ ID NO: 10), or SEQ ID NO: 22 (nucleobase sequence of SEQ ID NO: 11)
Each internucleotide linkage is a phosphorothioate linkage, all cytidines are 5′-methylcytidine and all nucleotides have a 2′-O-methyl base, e.g.: oligonucleotide with a nucleotide sequence comprising or consisting of the sequence of SEQ ID NO: 26 (nucleobase sequence of SEQ ID NO: 11), SEQ ID NO: 30 (nucleobase sequence of SEQ ID NO: 6), or SEQ ID NO: 34 (nucleobase sequence of SEQ ID NO: 7).

Accordingly, in a preferred embodiment, the oligonucleotide comprises a nucleotide sequence according to one of SEQ ID NOs: 12 to 22, 26, 30 and 34. In a more preferred embodiment, the nucleotide sequence of the oligonucleotide consists of a nucleotide sequence according to one of SEQ ID NOs: 12 to 22, 26, 30 and 34. In these embodiments, SEQ ID Nos: 17-19, 26, 30 and 34, are preferred; of these, SEQ ID NOs: 19, 26, 30 and 34 are preferred, in particular SEQ ID NO: 19.

Accordingly, in an embodiment, the oligonucleotide consists of SEQ ID NO: 19, 34, 17, 18, 30 or 26 and exhibit attractive therapeutic activity as demonstrated in the experimental part (see for example FIGS. 4, 5, 6). Within this embodiment, the oligonucleotide may be further modified as defined earlier herein.

Accordingly, in an embodiment, the oligonucleotide comprises SEQ ID NO:19 or 8. In an embodiment, such oligonucleotide has a length of 20 to 100 nucleotides. In an embodiment, the oligonucleotide consists of SEQ ID NO:19 or 8. Within this embodiment, the oligonucleotide may be further modified as defined earlier herein.

Accordingly, in an embodiment, the oligonucleotide comprises SEQ ID NO:34 or 18 or 7. In an embodiment, such oligonucleotide has a length of 20 to 100 nucleotides. In an embodiment, the oligonucleotide consists of SEQ ID NO:34 or 18 or 7. Within this embodiment, the oligonucleotide may be further modified as defined earlier herein.

Accordingly, in an embodiment, the oligonucleotide comprises SEQ ID NO:30 or 17 or 6. In an embodiment, such oligonucleotide has a length of 20 to 100 nucleotides. In an embodiment, the oligonucleotide consists of SEQ ID NO: 30 or 17 or 6. Within this embodiment, the oligonucleotide may be further modified as defined earlier herein.

Accordingly, in an embodiment, the oligonucleotide comprises SEQ ID NO:26 or 16 or 5. In an embodiment, such oligonucleotide has a length of 20 to 100 nucleotides. In an embodiment, the oligonucleotide consists of SEQ ID NO:26 or 16 or 5. Within this embodiment, the oligonucleotide may be further modified as defined earlier herein.

In an embodiment, there is provided an oligonucleotide, which comprises one of SEQ ID NO: 12, 13, 14, 15, 16, 17, 18, 19, 20, 21, 22 and has a length of 20 to 100 nucleotides or which consists of one of SEQ ID NO: 12, 13, 14, 15, 16, 17, 18, 19, 20, 21, 22. Within this embodiment, the oligonucleotide may be further modified as defined earlier herein.

In an embodiment, there is provided, an oligonucleotide, which comprises one of SEQ ID NO: 34, 30 or 26 and has a length of 20 to 100 nucleotides or which consists of one of SEQ ID NO: 34, 30 or 26. Within this embodiment, the oligonucleotide may be further modified as defined earlier herein.

In an embodiment, there is provided an oligonucleotide, which comprises SEQ ID NO: 19 or 34 and has a length of 20 to 100 nucleotides or which consists of SEQ ID NO: 19 or 34. Within this embodiment, the oligonucleotide may be further modified as defined earlier herein.

### Oligonucleotide, Conjugations

In some embodiments, the oligonucleotide is conjugated to one or more ligands. The ligand preferably is capable of targeting and/or delivering the oligonucleotide to (or into) a organ, a tissue and/or a cell, for example the kidney, kidney tissue, or kidney cell, or the bladder, bladder tissue, or bladder cell, in particular bladder epithelial cell.

Examples of ligands are e.g. peptides, vitamins, aptamers, carbohydrates or mixtures of carbohydrates (Han et al., Nature Communications, 2016, doi:10.1038/ncomms10981; Cao et al., Mol. Ther. Nucleic Acids, 2016, doi:10.1038/mtna.2016.46), proteins, small molecules, antibodies (or antigen-binding fragments thereof), polymers, drugs. Examples of carbohydrate conjugate group ligands are glucose, mannose, galactose, maltose, fructose, N-acetylgalactosamine (GaINac), glucosamine, N-acetylglucosamine, glucose-6-phosphate, mannose-6-phosphate, and maltotriose. Carbohydrates may be present in plurality, for example as end groups on dendritic or branched linker moieties that link the carbohydrates to the component of the composition. A carbohydrate can also be comprised in a carbohydrate cluster portion, such as a GaINAc cluster portion. A carbohydrate cluster portion can comprise a targeting moiety and, optionally, a conjugate linker. In some embodiments, the carbohydrate cluster portion comprises 1, 2, 3, 4, 5, 6, or more GaINAc groups. As used herein, “carbohydrate cluster” means a compound having one or more carbohydrate residues attached to a scaffold or linker group, (see, e.g., Maier et al., “Synthesis of Antisense Oligonucleotides Conjugated to a Multivalent Carbohydrate Cluster for Cellular Targeting,” Bioconjugate Chem., 2003, (14): 18-29; Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, (47): 5798-5808). In this context, “modified carbohydrate” means any carbohydrate having one or more chemical modifications relative to naturally occurring carbohydrates. As used herein, “carbohydrate derivative” means any compound which may be synthesized using a carbohydrate as a starting material or intermediate. As used herein, “carbohydrate” means a naturally occurring carbohydrate, a modified carbohydrate, or a carbohydrate derivative. Both types of excipients may be combined together into one single composition as identified herein. An example of a trivalent N-acetylglucosamine cluster is described in WO2017/062862 (Wave Life Sciences), which also describes a cluster of sulfonamide small molecules. An example of a single conjugate of the small molecule sertraline has also been described (Ferres-Coy et al., Mol. Psych. 2016, 21, 328), as well as conjugates of protein-binding small molecules, including ibuprofen (e.g. U.S. Pat. No. 6,656,730 ISIS/Ionis Pharmaceuticals), spermine (e.g. Noir et al., J. Am. Chem Soc. 2008, 130, 13500), anisamide (e.g. Nakagawa et al., J. Am. Chem. Soc. 2010, 132, 8848) and folate (e.g. Dohmen et al., Mol. Ther. Nucl. Acids 2012, 1, e7).

In an embodiment, the oligonucleotide is conjugated to lithocholic acid or eicosapentanoic acid.

In an embodiment, the oligonucleotide is conjugated (preferably via its 5′ or 3′ end, more preferably via its 3′ end) to a peptide, vitamin, aptamer, carbohydrate or mixtures of carbohydrates, protein, small molecule, antibody, polymer, drug, lithocholic acid, eicosapentanoic acid or a cholesterol moeity.

In a preferred embodiment, the oligonucleotide is conjugated to a small molecule, aptamer or antibody (or antigen-binding fragment thereof). Preferred antibodies (or antigen-binding fragments thereof) are specific to CD71 (transferrin receptor), described in e.g. WO2016/179257 (CytoMx), or against equilibrative nucleoside transporter (ENT), such as the 3E10 antibody, as described in e.g. Weisbart et al., Mol. Cancer Ther. 2012, 11, 1.

In another preferred embodiment, the oligonucleotide is conjugated to a GaINac moiety and/or to a cholesterol moeity. For example, the oligonucleotide is conjugated to the cholesterol moiety at its 3′ end and to the GaINac moiety at its 5′ end.

Generally, the conjugation is at the 5′ or the 3′ end of the oligonucleotide, preferably at the 3′ end.

### Oligonucleotide, Effects

Functionally, the oligonucleotide, optionally with modifications and/or conjugations as described above, can be characterized by being capable of exhibiting at least one of the following effects:
1) Modulating the splicing of T-antigen pre-mRNA,
2) Reducing the production of T-antigen mRNA,
3) Reducing the production of VP1 mRNA and preferably VP1 protein,
4) Inhibiting virus replication, preferably reducing the number of virus particles produced by a cell, and
5) Limiting the capacity of virus reinfection, i.e. limiting the capacity of an infected cell to produce infective viral particles.
   1) The modulation of the splicing of the T-antigen pre-mRNA may be assessed by monitoring the formation of a given splicing product of the pre-mRNA; a lower quantity of a given splicing product is the aim as it indicates inhibition of polyomavirus. A lower quantity may mean at least 5% lower than the quantity of the same splicing product at the onset of the treatment with the oligonucleotide, or at least 10%, 15%, 20%, 30%, 40%, 50%, 60%, 70%, 80%, 90%, or 95% lower. The assessment may be done using PCR.
   2) The reduction of T-antigen mRNA production may be by at least 10%, 20%, 30%, 40%, 50%, 60%, 70%, 80%, 90%, 95%, or 99% of the initial produced mRNA at the onset of the treatment with the oligonucleotide. Preferably, T-antigen mRNA is no longer detectable. mRNA production may be detected using techniques known to the skilled person, such as RT-PCR or Northern blotting.
   3) The reduction of VP1 mRNA and preferably VP1 protein production may be by at least 10%, 20%, 30%, 40%, 50%, 60%, 70%, 80%, 90%, 95%, or 99% of the initial produced mRNA (or protein) at the onset of the treatment with the oligonucleotide. Preferably, VP1 mRNA (or protein) is no longer detectable. mRNA and protein production may be detected using techniques known to the skilled person, including RT-PCR or Northern blotting for mRNA and Western blotting for protein.
   4) The virus replication may be inhibited in such a way that the amount of viral DNA is reduced by at least 10%, 20%, 30%, 40%, 50%, 60%, 70%, 80%, 90%, 95%, or 99% of the initial viral DNA at the onset of the treatment with the oligonucleotide. In an embodiment, the viral DNA is no longer detectable. The number of virus particles may be reduced by at least 10%, 20%, 30%, 40%, 50%, 60%, 70%, 80%, 90%, 95%, or 99% of the initial number of virus particles at the onset of the treatment with the oligonucleotide. In an embodiment, virus particles are no longer detectable. Virus replication or the number of virus particles produced may be assessed using techniques known to the skilled person. For example, virus replication or the number of virus particles (viral load) produced may be detected using PCR. The experimental part of the specification provides an exemplary method for detecting viral load.
   5) The limitation of the capacity of the virus to reinfect may be quantified such that the number of cells infected by virus produced from one infected cell treated with the oligonucleotide is reduced by at least 10%, 20%, 30%, 40%, 50%, 60%, 70%, 80%, 90%, 95%, or 99% compared to the number of cells infected by virus produced from one infected cell not treated with the oligonucleotide

Preferably, the oligonucleotide is capable of exhibiting at least effect 1. This effect on splicing is shown in the examples. Without being bound by theory, it is believed that the oligonucleotide inhibits usage of the splice site it targets. The resulting reduction in the production of large T-antigen impacts the expression of the capsid proteins and thereby the production of virus. Without being bound by theory, it is believed that the imbalance of T-antigen specific splice products induced by the oligonucleotide has a more pronounced effect on virus propagation than the reduction of T-antigen mRNA by RNAi-like approaches.

In one embodiment, the oligonucleotide exhibits at least (i) effect 1 and/or effect 2, and (ii) effect 4; or the oligonucleotide exhibits at least (i) effect 1 and/or effect 2, and (ii) effect 5. Preferably, the oligonucleotide exhibits at least (i) effect 1 and/or effect 2, (ii) effect 4, and (iii) effect 5.

In another embodiment, the oligonucleotide exhibits at least (i) effect 3, and (ii) effect 4; or the oligonucleotide exhibits at least (i) effect 3, and (ii) effect 5. Preferably, the oligonucleotide exhibits at least (i) effect 3, (ii) effect 4, and (iii) effect 5.

Therapeutic effects are defined below in context of the medical use.

### Polyomavirus

Within the context of the specification, a polyomavirus may be any polyomavirus. In an embodiment, the polyomavirus is a human polyomavirus, including all genera like the alpha, the beta and the delta genus. In a preferred embodiment, the polyomavirus is an alpha or a beta virus, preferably a beta virus. Non-limiting examples of human polyomaviruses are listed in Table 1 below. In a preferred embodiment, the polyomavirus is a BK polyomavirus (or BK virus, also referred to herein as BKPyV or BKV), a JC polyomavirus (or JC virus, also referred to herein as JCV) or a Merkel cell polyomavirus (MC polyomavirus, MC virus, or also referred to herein as MCV). In a particularly preferred embodiment, the polyomavirus is BK virus or JC virus, preferably BK virus.

TABLE 1
Examples of human polyomaviruses

| | | 3′ splice site | 5′ splice site |
|---|---|---|---|
| Abbreviation | Accession | target region | target region |
| BKPyV | NC_001538 | 4537-4596 | 4881-4940 |
| JCPyV | NC_001699 | 4397-4456 | 4741-4800 |
| KIPyV | NC_009238 | 4299-4358 | 4686-4745 |
| WUPyV | NC_009539 | 4477-4536 | 4876-4935 |
| MCPyV | NC_010277 | 4693-4752 | 5124-5183 |
| HPyV6 | NC_014406 | 4264-4323 | 4654-4713 |
| HPyV7 | NC_014407 | 4272-4331 | 4677-4736 |
| TSPyV | NC_014361 | 4352-4411 | 4765-4824 |
| HPyV9 | NC_015150 | 4408-4467 | 4760-4819 |
| MWPyV | NC_018102 | 4303-4362 | 4658-4717 |
| STLPyV | NC_020106 | 4159-4218 | 4504-4563 |
| HPyV12 | NC_020890 | 4392-4451 | 4791-4850 |
| NJPyV | NC_024118 | 4471-4530 | 4859-4918 |

In a second aspect, the invention relates to a vector comprising (i) an oligonucleotide as defined in the first aspect, (ii) the reverse complement of an oligonucleotide as defined in the first aspect, or (iii) DNA capable of being transcribed to an oligonucleotide as defined in the first aspect.

In a preferred embodiment, the vector is a nucleic acid vector. Accordingly, the vector according to (i) and (ii) is preferably an RNA vector, and the vector according to (iii) is preferably a DNA vector. Nucleic acid vectors include plasmid vectors, cosmid vectors, phage vectors such as lambda phage, and viral vector. Viral vectors are preferred and, in some embodiments, the viral vector may be selected from the group consisting of an adenoviral vector, an adeno-associated viral vector, a retroviral vector and a lentiviral vector. A preferred viral vector is an adeno-associated viral vector (AAV). Examples are AAV of serotype 1 (AAV1), AAV of serotype 2 (AAV2), AAV of serotype 3 (AAV3), AAV of serotype 4 (AAV4), AAV of serotype 5 (AAV5), AAV of serotype 6 (AAV6), AAV of serotype 7 (AAV7), AAV of serotype 8 (AAV8), AAV of serotype 9 (AAV9), AAV of serotype rh10 (AAVrh10), AAV of serotype rh8 (AAVrh8), AAV of serotype Cb4 (AAVCb4), AAV of serotype rh74 (AAVrh74), AAV of serotype DJ (AAVDJ), AAV of serotype 2/5 (AAV2/5), AAV of serotype 2/1 (AAV2/1), AAV of serotype 1/2 (AAV1/2), and AAV of serotype Anc80 (AAVAnc80). AAV2 is a preferred example.

Non-nucleic vectors, however, are also encompassed and include for example virus-like particles (VLPs) or “VLP” refers to a non-replicating, empty viral shell. VLPs are generally composed of one or more viral proteins, such as, but not limited to those proteins referred to as capsid, coat, shell, surface and/or envelope proteins. They contain functional viral proteins responsible for cell penetration by the virus, which ensures efficient cell entry. Methods for producing particular VLPs are known in the art.

In a third aspect, the invention relates to a composition comprising an oligonucleotide as defined in the first aspect or a vector as defined in the second aspect. In a preferred embodiment, this composition is a pharmaceutical composition.

The pharmaceutically composition preferably comprises one or more pharmaceutically acceptable excipients, such as a filler, preservative, solubilizer, carrier, diluent, excipient, salt, adjuvant and/or solvent, as described for instance in Remington: The Science and Practice of Pharmacy, 20th Edition. Baltimore, MD: Lippincott Williams & Wilkins, 2000.

An excipient may enhance the stability, solubility, absorption, bioavailability, activity, pharmacokinetics, pharmacodynamics, cellular uptake, and intracellular trafficking of the oligonucleotide, in particular it may be an excipient capable of forming complexes, nanoparticles, microparticles, nanotubes, nanogels, hydrogels, poloxamers or pluronics, polymersomes, colloids, microbubbles, vesicles, micelles, lipoplexes, and/or liposomes. Examples of nanoparticles include polymeric nanoparticles, (mixed) metal nanoparticles, carbon nanoparticles, gold nanoparticles, magnetic nanoparticles, silica nanoparticles, lipid nanoparticles, sugar particles, protein nanoparticles and peptide nanoparticles. An example of the combination of nanoparticles and oligonucleotides is spherical nucleic acid (SNA), as in e.g. Barnaby et al. Cancer Treat. Res. 2015, 166, 23.

A preferred excipient is a targeting excipient, which is capable of targeting and/or delivering the oligonucleotide to (or into) an organ, a tissue and/or a cell, for example the kidney, kidney tissue, or kidney cell, or the bladder, bladder tissue, or bladder cell, in particular bladder epithelial cell. Many of these excipients are known in the art (e.g. see Bruno, K. et al, (2011), Adv. Drug. Deliv. Rev, 63(13): 210-1226), examples include polymers (e.g. polyethyleneimine (PEI), polypropyleneimine (PPI), dextran derivatives, butylcyanoacrylate (PBCA), hexylcyanoacrylate (PHCA), poly(lactic-co-glycolic acid) (PLGA), polyamines (e.g. spermine, spermidine, putrescine, cadaverine), chitosan, poly(amido amines) (PAMAM), poly(ester amine), polyvinyl ether, polyvinyl pyrrolidone (PVP), polyethylene glycol (PEG) cyclodextrins, hyaluronic acid, colominic acid, and derivatives thereof), dendrimers (e.g. poly(amidoamine)), lipids {e.g. 1,2-dioleoyl-3-dimethylammonium propane (DODAP), dioleoyldimethylammonium chloride (DODAC), phosphatidylcholine derivatives [e.g. 1,2-distearoyl-sn-glycero-3-phosphocholine (DSPC)], lyso-phosphatidylcholine derivaties [e.g. 1-stearoyl-2-lyso-sn-glycero-3-phosphocholine (S-LysoPC)], sphingomyelin, 2-{3-[Bis-(3-amino-propyl)-amino]-propylamino}-N-ditetradecyl carbamoyl methylacetamide (RPR209120), phosphoglycerol derivatives [e.g. 1,2-dipalmitoyl-sn-glycero-3-phosphoglycerol, sodium salt (DPPG-Na), phosphatidic acid derivatives [1,2-distearoyl-sn-glycero-3-phosphatidic acid, sodium salt (DSPA), phosphatidylethanolamine derivatives [e.g. dioleoyl-L-R-phosphatidylethanolamine (DOPE), 1,2-distearoyl-sn-glycero-3-phosphoethanolamine (DSPE),2-diphytanoyl-sn-glycero-3-phosphoethanolamine (DPhyPE),], N-[1-(2,3-dioleoyloxy)propyl]-N,N,N-trimethylammonium (DOTAP), N-[1-(2,3-dioleyloxy)propyl]-N,N,N-trimethylammonium (DOTMA), 1,3-di-oleoyloxy-2-(6-carboxy-spermyl)-propylamide (DOSPER), (1,2-dimyristyloxypropyl-3-dimethylhydroxy ethyl ammonium (DMRIE), (N1-cholesteryloxycarbonyl-3,7-diazanonane-1,9-diamine (CDAN), dimethyldioctadecylammonium bromide (DDAB), 1-palmitoyl-2-oleoyl-sn-glycerol-3-phosphocholine (POPC), (b-L-Arginyl-2,3-L-diaminopropionic acid-N-palmityl-N-oleyl-amide trihydrochloride (AtuFECT01), N,N-dimethyl-3-aminopropane derivatives [e.g. 1,2-distearyloxy-N,N-dimethyl-3-aminopropane (DSDMA), 1,2-dioleyloxy-N,N-dimethyl-3-aminopropane (DoDMA), 1,2-Dilinoleyloxy-N,N-3-dimethylaminopropane (DLinDMA), 2,2-dilinoleyl-4-dimethylaminomethyl[1,3]-dioxolane (DLin-K-DMA), phosphatidylserine derivatives [1,2-dioleyl-sn-glycero-3-phospho-L-serine, sodium salt (DOPS)], proteins (e.g. albumin, gelatins, atelocollagen), and linear or cyclic peptides (e.g. protamine, PepFects, NickFects, polyarginine, polylysine, CADY, MPG, cell-penetrating peptides (CPPs), targeting peptides, cell-translocating peptides, endosomal escape peptides). Examples of such peptides have been described, e.g. muscle targeting peptides (e.g. Jirka et al., Nucl. Acid Ther. 2014, 24, 25), CPPs (e.g. Pip series, including WO2013/030569, and oligoarginine series, e.g. U.S. Pat. No. 9,161,948 (Sarepta), WO2016/187425 (Sarepta), and M12 peptide in e.g. Gao et al., Mol. Ther. 2014, 22, 1333), or blood-brain barrier (BBB) crossing peptides such as (branched) ApoE derivatives (Shabanpoor et al., Nucl. Acids Ther. 2017, 27, 130). Carbohydrates and carbohydrate clusters as described above with respect to oligonucleotide conjugations are also suitable for use as an excipient.

The pharmaceutical composition is formulated to comprise a pharmaceutically effective amount of the oligonucleotide. It can be formulated for administration via the topical, systemic and/or parenteral route, for example intravenous, subcutaneous, intraperitoneal, intrathecal, intramuscular, ocular, nasal, urogenital, intradermal, dermal, enteral, intravitreal, intracavernous, intracerebral, intrathecal, epidural or oral route.

Preferably, the pharmaceutical composition is formulated as an emulsion, suspension, pill, tablet, capsule or soft-gel for oral delivery, or in the form of aerosol or dry powder for delivery to the respiratory tract and lungs.

In an embodiment, the pharmaceutical composition further comprises a further active ingredient for the treatment of a polyomavirus infection.

In a fourth aspect, the invention relates to an oligonucleotide as defined in the first aspect, a vector as defined in the second aspect or a pharmaceutical composition as defined in the third aspect for use as a medicament, in particular for use in treating a polyomavirus infection in a subject.

The oligonucleotide, the vector and the pharmaceutical composition are collectively referred to hereinafter as “medicament” for the sake of brevity.

Thus, while the medicament is generally for use in treating a polyomavirus infection in a subject, it is preferably also for use in treating a disease associated with a polyomavirus. The meaning of polyomavirus is defined above with regard to the first aspect. “Diseases associated with a polyomavirus” include cystitis (in particular haemorrhagic cystitis e.g. in recipients of bone marrow transplantation), urethritis, interstitial nephritis (also known as nephropathy; in particular transplant nephropathy), and progressive multifocal leukoencephalopathy (in particular in immunocompromised subjects).

The term “infection” as used herein refers to a viral infection, i.e. to the entry of a virus into at least one cell of a host and its replication within the at least one cell. An infection may be acute (i.e. active) or, as e.g. in the case of polyomavirus, also latent (i.e. inactive, hidden, dormant). In an acute infection, the virus is replicating, infects cells and potentially causes symptoms, whereas in a latent infection, the virus does not replicate independent from the host cell genome and infect further cells, it rather “hides” in a cell. A latent infection can be interrupted by acute infections in which the hidden virus starts replicating and infecting further cells. In the case of a pre-existing latent infection, the use in treating an infection preferably relates to preventing an acute infection by preventing the hidden virus from infecting further cells, i.e. from spreading. In other words, this can be described as a treatment of a latent infection, wherein the treatment is not necessarily curative (but keeps the virus in check). In other words, the subject treated with the medicament may be asymptomatic. In an embodiment, the subject treated with the medicament is asymptomatic and seropositive. In another embodiment, the subject treated with the medicament is asymptomatic and seronegative.

The medicament is administered in a therapeutically effective amount. The induction of such a therapeutic effect may be assessed in vitro (i.e. cell free or in a cell) or in vivo (i.e. in an animal such as an animal model or in a patient). It may be assessed at the molecular level and/or at the cellular level.

“Therapeutic effect” inter alia refers to effects 1) to 5) defined with respect to the first aspect of the invention above, i.e. these effects are referred to also as therapeutic effects 1) to 5), respectively. Further therapeutic effects are:
6) Preventing a cellular effect associated with an infection,
7) Alleviating a cellular effect associated with the infection,
8) Preventing disease in an infected subject, and/or
9) Slowing down, preferably stopping, more preferably reversing disease progression.
   6) and 7): The cellular effect associated with the infection may be cell death (e.g. by apoptosis or cell lysis), the alleviation of which would be a decrease in cell death. Such a decrease of cell death may be by at least 5% compared to the level of cell death at the onset of the treatment. Preferably, a decrease of cell death means at least 10%, even more preferably at least 20%, at least 30%, at least 40%, at least 50%, at least 70%, at least 80%, at least 90%, or 100%. Cell death can be determined by detecting cell death directly or by detecting cell survival, e.g. by harvesting a population of cells and (immune)staining for cell cycle markers, determining the cell cycle phase(s) with appropriate markers by FACS analysis or by PCR for expression levels of cell cycle RNAs.
   8) and 9): Whether disease is prevented or progression is slowed down, stopped or reversed can be determined by assessing symptoms or parameters of the disease associated with polyomavirus infection. Such symptoms are known in the art. For example, to assess the progression of nephropathy, parameters such as glomerular filtration rate or creatinine levels can be determined.

Additional parameters that may be assessed under 8) or 9) as a (molecular) marker of the presence or of the status or of the progression of the disease or condition include: cyclin E2 (CCNE2), cell division cycle 6 (CDC6), cyclin E2 (CCNA2), E2F transcription factor 8 (E2F8), survivin (BIRC5), RAD51-associated protein-1 (RAD51AP1), BRCA1-interacting protein C-terminal helicase 1 (BRIP1) and apolipoprotein B mRNA-editing enzyme 3B (APOBEC3B).

The treatment with the oligonucleotide as defined herein may increase a downregulation or decrease of at least one of these genes (Abend, J. et al. (2010) Global effects of BKV infection of gene expression in human primary epithelial cells; 397 (1): 73). In an embodiment, the downregulation or decrease is induced following BKV infection or reinfection of cells.

In an embodiment, the downregulation or decrease is of at least 10%, 20%, 30%, 40%, 50%, 60%, 70%, 80%, 90%, 95%, 99% of the initial expression level of said gene at the onset of the treatment with the oligonucleotide. In an embodiment, the expression is no longer detectable. Expression may be assessed using techniques known to the skilled person. In an embodiment, expression is assessed using northern blot or PCR.

Accordingly in an embodiment, an oligonucleotide, a vector or a pharmaceutical composition as defined herein are for use as a medicament, preferably for treating a polyomavirus infection in a subject, wherein said oligonucleotide, vector or pharmaceutical composition is capable of exhibiting at least one of the following effects:
1) Modulating the splicing of T-antigen pre-mRNA,
2) Reducing the production of T-antigen mRNA,
3) Reducing the production of VP1 mRNA and preferably VP1 protein,
4) Inhibiting virus replication,
5) Limiting the capacity of virus reinfection, i.e. limiting the capacity of an infected cell to produce infective viral particles,
6) Preventing a cellular effect associated with an infection,
7) Alleviating a cellular effect associated with the infection,
8) Preventing disease in an infected subject, and
9) Slowing down, preferably stopping, more preferably reversing disease progression.
   In an embodiment, an oligonucleotide, a vector or a pharmaceutical composition as defined herein are for use as a medicament, preferably for treating a polyomavirus infection in a subject, wherein the disease has been prevented (8) or progression slowed down, stopped or reversed (9) when at least one parameters of the disease associated with said polyomavirus infection has been reduced, said parameters being selected from: glomerular filtration rate, creatinine levels, cyclin E2 (CCNE2), cell division cycle 6 (CDC6), cyclin E2 (CCNA2), E2F transcription factor 8 (E2F8), survivin (BIRC5), RAD51-associated protein-1 (RAD51AP1), BRCA1-interacting protein C-terminal helicase 1 (BRIP1) and apolipoprotein B mRNA-editing enzyme 3B (APOBEC3B).

It is envisaged that the therapeutic effect includes at least one of therapeutic effects 1) to 5), as defined above with regard to the first aspect, and preferably also at least one of therapeutic effects 6) to 9). An effect is assessed by comparison to the state at the onset of the treatment.

The subject is preferably a human. Usually, polyomavirus infects and replicate in a host that is permissive to it, i.e. a host that allows a virus to circumvent its defenses and to replicate, such as an immunocompromised host. This can also be described by the term immunodeficiency, which is in a state in which the immune system's ability to fight infectious disease and cancer is compromised or entirely absent. This state may be temporary or permanent. In one embodiment, immunodeficiency is acquired (“secondary”), usually due to extrinsic factors that affect the patient's immune system. Examples of these extrinsic factors include HIV infection, (extremes of) age, and environmental factors, such as malnutrition. Immunodeficiency can also be induced by drugs, such as glucocorticoids, cytostatics, antibodies, and compounds that act upon immunophilins (such as calcineurin inhibitors, belatacept (an immunoglobulin like molecule that has the extracellular domain of CTLA-4) and similar molecules). This can be a desired effect such as in organ transplant surgery as an anti-rejection measure and in subjects suffering from an overactive immune system, as in autoimmune diseases. However, sometimes this desired effect has the additional effect of reducing the subject's ability to combat polyomavirus infection. A subject who has an immunodeficiency of any kind is said to be immunocompromised. In another embodiment, the immunodeficiency is inherited. For example, it may be a genetic defect, usually recessive, affecting B- or T-cell lymphocyte development, phagocytic cells, complement, cytokines or its receptors, antibodies or other components of the innate or adaptive immune system.

Thus, in an embodiment, the subject is immunocompromised, e.g. due to one of the factors above and/or another infection, a cancer, or the use of a drug administered to the subject to treat another condition or disease. In one embodiment, the other condition is organ, tissue or cell (in particular kidney) transplantation. Accordingly, in one embodiment, the subject is the recipient of an organ, tissue or cell transplant (herein also referred collectively as “transplant”).

In a fifth aspect, the invention relates to ex-vivo methods, including an ex-vivo method of inhibiting polyomavirus replication in a cell, and an ex-vivo method of producing a transplant.

The ex-vivo method of inhibiting polyomavirus replication in a cell comprises (i) providing a cell that infected with polyomavirus, and contacting the cell with an oligonucleotide as defined in the first aspect, a vector as defined in the second aspect or a pharmaceutical composition as defined in the third aspect; or (ii) providing a cell comprising an oligonucleotide as defined in the first aspect, a vector as defined in the second aspect or a pharmaceutical composition as defined in the third aspect, and contacting the cell with polyomavirus. The ex-vivo method of producing a transplant comprises providing a donor organ, tissue or cell(s) (preferably comprising kidney cells), and contacting cells of the donor organ, tissue or cell(s) with an oligonucleotide as defined in the first aspect, a vector as defined in the second aspect or a pharmaceutical composition as defined in the third aspect.

### General Definitions

Unless stated otherwise, all technical and scientific terms used herein have the same meaning as customarily and ordinarily understood by a person of ordinary skill in the art to which this invention belongs, and read in view of this disclosure.

A “nucleobase”, sometimes called a base, is generally adenine, cytosine, guanine, thymine, or uracil, or a derivative thereof. Cytosine, thymine, and uracil are pyrimidine bases, and are generally linked to the scaffold through their 1-nitrogen. Adenine and guanine are purine bases, and are generally linked to the scaffold through their 9-nitrogen. RNA nucleobases as referred to herein are adenine, cytosine, guanine, and uracil.

Unless indicated otherwise, a “nucleotide” referred to herein stands for an RNA nucleotide, preferably a naturally occurring RNA nucleotide. The most common naturally occurring nucleotides in RNA are adenosine monophosphate, cytidine monophosphate, guanosine monophosphate, and uridine monophosphate. These consist of a pentose sugar ribose, a 5′-linked phosphate group which is linked via a phosphate ester, and a 1′-linked base. The sugar connects the base and the phosphate, and is therefore often referred to as the scaffold of the nucleotide. A modification in the pentose sugar is therefore often referred to as a scaffold modification. A sugar modification may therefore be called a scaffold modification. For severe modifications, the original pentose sugar might be replaced in its entirety by another moiety that similarly connects the base and the phosphate. It is therefore understood that while a pentose sugar is often a scaffold, a scaffold is not necessarily a pentose sugar. A nucleotide is generally connected to neighbouring nucleotides through condensation of its 5′-phosphate moiety to the 3′-hydroxyl moiety of the neighbouring nucleotide monomer. Similarly, its 3′-hydroxyl moiety is generally connected to the 5′-phosphate of a neighbouring nucleotide monomer. This forms phosphodiester bonds. The phosphodiesters and the scaffold form an alternating copolymer. The bases are grafted to this copolymer, namely to the scaffold moieties. Because of this characteristic, the alternating copolymer formed by linked monomers of an oligonucleotide is often called the backbone of the oligonucleotide. Because the phosphodiester bonds connect neighbouring monomers together, they are often referred to as backbone linkages. It is understood that when a phosphate group is modified so that it is instead an analogous moiety such as a phosphorothioate, such a moiety is still referred to as the backbone linkage of the monomer. This is referred to as a backbone linkage modification. In general terms, the backbone of an oligonucleotide is, thus, comprised of alternating scaffolds and backbone linkages.

The term “specifically binds” in the context of oligonucleotides means that the oligonucleotide is capable of annealing to its target. The term “binds” may be replaced by “hybridizes”, “targets”, “is directed against”, “is antisense to” or is “complementary to”. The binding of the oligonucleotide to its target pre-mRNA may be assessed using EMSA (Electrophoretic Mobility Shift Assay) using the oligonucleotide and incubating it with a polyomavirus RNA.

The term “annealing”, when used with respect to an oligonucleotide, is to be understood as a bond of an oligonucleotide to an at least substantially complementary sequence with respect to base pairing involving hydrogen bonding, which may be Watson-Crick, Hoogsteen or reversed Hoogsteen Stringent hybridization conditions involve hybridizing at 68° C. in 5×SSC/5×Denhardt's solution/1.0% SDS, and washing in 0.2×SSC/0.1% SDS at room temperature, or involve the art-recognized equivalent thereof (e.g., conditions in which a hybridization is carried out at 60° C. in 2.5×SSC buffer, followed by several washing steps at 37° C. in a low buffer concentration, and remains stable). Moderate conditions involve washing in 3×SSC at 42° C., or the art-recognized equivalent thereof. The parameters of salt concentration and temperature can be varied to achieve the optimal level of identity between the probe and the target nucleic acid. Guidance regarding such conditions is available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

Hybridization of complementary strands typically improves with the length of the sequence. Specific hybridization of two strands is accomplished with a contiguous stretch of 12, 13, 14, 15, 16, 17, 18, 19, or 20 (preferably 18, 19 or 20) or more complementary nucleobases. The sequence of an oligonucleotide can be, but need not necessarily be, 100% complementary to that of its target sequence to hybridize. Moreover, an oligonucleotide may hybridize over one or more segments such that intervening or adjacent segments are not involved in the hybridization event. For example, the oligonucleotide comprises at least 70%, 75%, 80%, 85%, 90%, 95%, 96%, 97%, 98% or 99% sequence complementarity to a target region within the pre-mRNA. For example, an oligonucleotide in which 18 of 20 nucleobases of the oligonucleotide are complementary to a target region, and would therefore specifically hybridize, would represent 90 percent complementarity. When an oligonucleotide of 18 nucleotides has a sequence that is the reverse complement of a contiguous stretch of at least 12 nucleobases of a polyomavirus large T-antigen pre-mRNA, the remaining 6 complementary nucleobases may be clustered with the 12 or not be contiguous with the 12. Percent complementarity of an oligonucleotide with a region of a target pre-mRNA can be determined routinely using BLAST programs (basic local alignment search tools) and PowerBLAST programs known in the art (Altschul et al, J. Mol. Biol., 1990, 215, 403-410; Zhang and Madden, Genome Res., 1997, 7,649-656).

Preferably, an oligonucleotide is capable of annealing to a target if contiguous stretch of at least 12 nucleobases of a sequence is at least substantially complementary to the target. “Substantially complementary” means substantially identical to the reverse complement of the target sequence. “Substantially identical” means that an oligonucleotide does not need to be 100% identical to a reference sequence but can comprise mismatches and/or spacers as defined herein. It is preferred that a substantially identical oligonucleotide, if not 100% identical, comprises 1 to 3, i.e. 1, 2 or 3 mismatches and/or spacers, preferably one mismatch or spacer per oligonucleotide, such that the intended annealing does not fail due to the mismatches and/or spacers. To enable annealing despite mismatches and/or spacers, it is preferred that an oligonucleotide does not comprise more than 1 mismatch per 10 nucleotides (rounded up if the first decimal is 5 or higher, otherwise rounded down) of the oligonucleotide. The term “spacer” as used herein refers to a non-nucleotide spacer molecule, which increases, when joining two nucleotides, the distance between the two nucleotides to about the distance of one nucleotide (i.e. the distance the two nucleotides would be apart if they were joined by a third nucleotide). Non-limiting examples for spacers are Inosine, d-Uracil, halogenated bases, Amino-dT, C3, C12, Spacer 9, Spacer 18, and dSpacer.

The oligonucleotide may comprise a 3′ and/or a 5′ overhang, i.e. a contiguous stretch of nucleobases, which is not substantially complementary to the target region. Such overhangs do not prevent the oligonucleotide from specifically binding to its target region.

The terms “complement” and “reverse complement” are used similarly, in that a complement to a target nucleic acid has a sequence which is the reverse complement of the target sequence. Therein, “reverse” reflects that a sequence is usually recited starting with the 5′ end of the nucleic acid.

“Pre-mRNA” or “precursor mRNA” is an immature single strand of messenger ribonucleic acid (mRNA). Polyomavirus T-antigen pre-mRNA is synthesized from a polyomavirus DNA template in the cell nucleus by transcription. The pre-mRNA contains one or more introns that are spliced out during maturation of the pre-mRNA into mRNA. The splicing process removes introns from transcripts and joins exons together. Introns are typically flanked by a donor site (5′ end of the intron) and an acceptor site (3′ end of the intron). The splice sites are required for splicing and typically include an almost invariant sequence GU at the 5′ end of the intron and a splice acceptor site at the 3′ end of the intron with a usually invariant AG sequence. The GU and AG sequence and the intervening sequence are spliced out of the pre-mRNA. A characteristic of polyomavirus T-antigen pre-mRNA is that it can be alternatively spliced or not spliced leading to the generation of at least two and often 3, 4 or 5 differently spliced mRNAs. Virus propagation is dependent on the availability of the virus genome, the presence of virus proteins, the cellular machinery and particularly the delicate interplay between the various stages and components. The splicing process of virus RNAs is an important method for regulating the virus propagation process, and influences the level and likely also the timing of certain products being formed in the cells. The term “splice site” refers to a sequence (“splice sequence”) defining the splice locus.

“Capable of being transcribed” as used herein means that a DNA nucleobase sequence is identical to an RNA nucleobase sequence with the exception that DNA has thymine at all uracil positions of the RNA.

The “onset of the treatment with the oligonucleotide” referred to herein with regard to effects of the oligonucleotide means a treatment comprising contacting an infected cell with the oligonucleotide. The treatment may be an in vitro (e.g. cell culture) or an in vivo (non-human animal or human) treatment.

“Ex-vivo”, in its broadest sense, means in vitro. In a preferred embodiment, it means refers to experimentation or measurements done in or on tissue from an organism in an external environment with minimal alteration of natural conditions.

### EXAMPLES

### Example 1

### Results

ASO Nanowalk and Candidate Modification Previously, we developed antisense oligonucleotides (ASOs) that target the exon-intron junctions of the BKV early coding region pre-mRNA that leads to the generation of large T antigen, small t antigen and truncated T antigen mRNAs (WO 2019/168402). These studies yielded several ASOs that demonstrated an ability to attenuate expression of 1) large T antigen and VP1 mRNA; 2) VP1 protein; and 3) viral DNA production. Within the BKV early coding region pre-mRNA, the most 5′ ASO possessed but 1 nucleobase in the intronic region of large T antigen sequence, while the most 3′ ASO possessed but 1 nucleobase in the exonic portion of large T antigen exon 1. As shown in FIG. 2, the positioning of ASOs consistently moved 2 nucleotides downstream of SEQ ID NO: 1 in a 5′ to 3′ orientation, allowing for effective tiling of the entire exon 1—intronic junction with the herein described SEQ ID NOs: 1 to 11 (RNA sequences) and corresponding 2′-OMe modified ASOs (SEQ ID NOs: 12 to 22). Hence, for these studies, 11 additional ASOs (SEQ ID NOs: 12 to 22) were generated and screened for activity in human PTECs.

Surprisingly, several of these new 2′-OMe modified ASOs (FIG. 2) displayed superior targeting activity towards the large T antigen exon 1 and/or intron donor site (FIG. 3-4).

This insight was gained by performing screening studies in human proximal tubule epithelial cells (hPTECs), which resulted in potent efficacy against 1) TAg and VP1 mRNA; 2) VP1 protein; and 3) virus particle production, as shown in FIG. 3. Subsequently, we extensively tested various modifications to these lead candidates by replacing 2′OMe-cytosines with 5-methylcytidines (SEQ ID NOs: 26, 30, 34 and 38) and various percentages of 2′-methoxy-ethyl (MOE) modified oligonucleotides (SEQ ID NOs: 23, 24, 25, 27, 28, 29, 31, 32, 33, 35, 36, 37). These studies consistently indicated that ASOs possessing 2′-O-methyl modifications yielded the greatest reductions in VP1 protein and BKV DNA replication (FIG. 4). These data, along with our previous studies in which we varied the amount of phosphorothioates incorporated into the ASO backbone prompted us to continue with ASO that are fully 2′-OMe and phosphorothioated.

### BKV-Targeting ASOs Limit ‘Reinfection’

Based on the observed reductions in viral DNA produced in response to treatment with our BKV-targeting ASOs, we elected to undertake ‘reinfection’ studies. For this, we treated hPTECs with SEQ ID NO: 26, SEQ ID NO: 17, SEQ ID NO: 34 or SEQ ID NO: 19 and 16 h later infected these with BKV. The cells were incubated for 7 days in culture medium, after which the conditioned medium, containing active and inactive viral particles, was harvested. The conditioned medium was subsequently 10× diluted and placed on untreated hPTECs. After 72 h, cells were fixed and immunostained for large T antigen positivity (FIG. 5), revealing markedly lower numbers of large T antigen positive cells following treatment with SEQ ID NO: 19 (and HYB_03 (SEQ ID NO: 68)) relative to control cells (left panel). Furthermore, the degree of positivity upon treatment with SEQ ID NO: 19 (and HYB_03 (SEQ ID NO: 68)) was lower than that observed for control cells (as well as SEQ ID NO: 26 and SEQ ID NO: 17 treated cells). As shown in FIG. 6, assessment of TAg and VP1 mRNA levels revealed the greatest degree of reduction following pre-treatment with SEQ ID NO: 19, as well as VP1 protein. Viral DNA levels and the number of reinfected cells were markedly reduced upon treatment with SEQ ID NO: 19.

### BKV-Targeting ASOs Induce Aberrant Splicing of BKV Early Coding Region Pre-mRNA

We subsequently investigated in pRPc cells the ability of our BKV-targeting ASOs to disrupt the appropriate splicing of the BKV early coding region pre-mRNA. For this, we administered our ASOs to pRPc cells and harvested RNA 24 hours post-transfection. As shown in FIG. 7, these studies revealed that lipofectamine and scramble-control ASO-treated cells did not reveal altered splicing profiles (lanes 1-3). In contrast, HYB_03 (SEQ ID NO: 68) and SEQ ID NO: 19 both resulted in diminished large T antigen mRNA levels (product 1) and a shift to slightly increased small t antigen mRNA (product 3, lanes 4-5). Simultaneously, clear evidence is evident of aberrantly spliced products between product 1 and 3, as well as a mRNA product smaller than product 1. Furthermore, our introduction of mismatch nucleobases into SEQ ID NO: 19 at position 10 (SEQ ID NO: 39), position 5 and 10 (SEQ ID NO: 40) and position 5, 10 and 15 (SEQ ID NO:41) resulted in gradual titration of the splicing effect (FIG. 7, lanes 6-8). These studies clearly show that SEQ ID NO: 19 mediates abrogation of large T antigen mRNA in a hybridization-dependent fashion. Next, we similarly assessed whether SEQ ID NO: 19 designed as a gapmer (RNA-DNA-RNA hybrid antisense oligonucleotide) could lead to similar or greater levels of large T antigen (pre-)mRNA degradation in an RNaseH-dependent fashion. As shown in FIG. 8 (lane 3), a SEQ ID NO: 19 GapmeR (SEQ ID NO: 42) did not yield potent reductions in large T antigen mRNA levels (product 1), while additional GapmeRs targeting exon 1 the early coding region pre-mRNA (SEQ ID NOs: 42 to 45) similarly did not result in meaningful reductions in large T or small t antigen mRNA expression levels.

Exposure of PBMCs to BKV-Targeting ASOs does not Impact Viability

Next, we assessed whether exposure to increasing levels of BKV-targeting ASOs could impact cell viability, whereby peripheral blood derived monocytes (PBMCs) were exposed to 1 or 10 μM ASO for 48 h. As shown in FIG. 9, multiple controls for cell viability were introduced, including exposure to 65° C. for 30 minutes prior to culture for 48 h (triggering decreased cell viability) as well as treatment with R848 (a TLR3 agonist) that enhances PBMC viability (Doyle S. L., et al (2007), J. Biol Chem. 282 (51): 36953-36960). Exposure to SEQ ID NO: 30, SEQ ID NO: 18, SEQ ID NO: 34 or SEQ ID NO: 19, nor increasing concentrations thereof, did not appear to impact PBMC viability.

### Exposure of PBMCs to BKV-Targeting ASOs Results in Mild Activation of Cytokine Production

To control for potential activation of pro-inflammatory cytokine expression by monocytes and macrophages in response to exposure to increasing levels of BKV-targeting ASOs, we treated PBMCs with 1 or 10 μM ASO for 48 h and harvested culture medium for multiplex ELISA analysis of cytokine production. As shown in FIGS. 10 and 11, exposure to SEQ ID NO:30, SEQ ID NO:18, SEQ ID NO:34 or SEQ ID NO:19 did not reveal striking increases in pro- nor anti-inflammatory cytokine production.

### Blood Coagulation Time is not Affected by BKV-Targeting ASOs

ASOs have, on occasion, been described to impact the time required for blood to physiologically coagulate. This could be detrimental in patients receiving an oligonucleotide-based therapy, including a BKV-targeting ASO post-kidney transplantation. Therefore, we elected to assess whether exposure of blood to increasing concentrations of our BKV-targeting ASOs could increase the coagulation time by determining the activated partial thromboplastin time (aPTT). For this, human plasma was collected and exposed to SEQ ID NO: 30, SEQ ID NO: 18, SEQ ID NO: 34 or SEQ ID NO: 19 at a concentration of 1 and 10 μM. As shown in FIG. 12, coagulation time was slightly augmented upon exposure to higher concentrations of BKV-targeting ASOs,

### BKV-Targeting ASOs Display Excellent Biodistribution to the Kidney

Since BKV primarily resides in the tubular epithelial cells of the kidney (and bladder epithelium), we performed a four-time intravenous dosing study with five candidate ASOs (concentration of 40 mg/kg on day 0, 3, 7 and 10 with sacrifice on day 14, FIG. 13 left panel) to examine the biodistribution of our BKV-targeting ASOs to the kidney, and in particular the proximal and distal tubule epithelial cells. As shown in FIG. 13 (left and middle image), the oligonucleotide having SEQ ID NO: 19 clearly displayed uptake in the proximal tubules, with weak staining observed in the distal compartment of the nephronic segment (FIG. 13, right image). We have developed a hybridization ELISA (hELISA) probe to determine SEQ ID NO: 19 levels in various organs in a quantitative fashion. As shown in FIG. 14, these studies revealed the kidney to be the primary organ of SEQ ID NO: 19 uptake (on a per gram basis per organ), with approximately 2.75-fold greater uptake than the liver and 4-fold greater uptake that the spleen. These extrarenal organs are widely considered to be excellent reservoirs for ASO uptake, clearly revealing the superior distribution of ASOs to the kidney.

### BKV-Targeting ASO Treatment Regimens do not Affect Kidney or Liver Function

Within the context of our four-time intravenous dosing study (concentration of 40 mg/kg on day 0, 3, 7 and 10 with sacrifice on day 14), we did not observe any overt signs of kidney or liver toxicity/damage. As shown in FIG. 15, serum levels of creatinine and blood urea nitrogen, as well as urine albumin levels were well within normal range following treatment with SEQ ID NO: 30, SEQ ID NO: 18, SEQ ID NO: 34 or SEQ ID NO: 19. Similarly, serum levels of aspartate aminotransferase and alanine aminotransferase, and the ratio thereof, did not yield any evidence of liver toxicity following 4× administration of SEQ ID NO: 30, SEQ ID NO: 18, SEQ ID NO: 34 or SEQ ID NO: 19.

### BKV-Targeting ASO Treatment Regimens do not Result in Overt Kidney Injury

Immunohistochemical staining for evidence of kidney injury following four-times administration of SEQ ID NO: 30, SEQ ID NO: 18, SEQ ID NO: 34 or SEQ ID NO: 19 did not reveal signs of overt/acute kidney injury. Initial assessments of KIM-1 and picrosirius red staining did not pinpoint kidney injury nor collagen deposition (see FIG. 16).

### Material and Methods

### Accessions Used for Phylogenetic Analysis

Complete genomic sequences of BK polyomavirus isolates were downloaded from the publicly available NCBI database. From these records, only the isolates reporting a complete genome were used for the conservation of the splice sites in Large T antigen. The Dunlop strain was used as a reference genome. Isolates “MM” and “FNL-9” were removed due to a large deletion in the intron or duplication overlapping the acceptor splice site respectively. Accession numbers of the 245 unique genomic sequences are provided below: AB211369.1; AB211370.1; AB211371.1; AB211372.1; AB211373.1; AB211374.1; AB211375.1; AB211376.1; AB211377.1; AB211378.1; AB211379.1; AB211381.1; AB211382.1; AB211383.1; AB211384.1; AB211385.1; AB211386.1; AB211387.1; AB211388.1; AB211389.1; AB211390.1; AB211391.1; AB213487.1; AB217917.1; AB217918.1; AB217919.1; AB217920.1; AB217921.1; AB260028.1; AB260029.1; AB260030.1; AB260031.1; AB260032.1; AB260033.1; AB263912.1; AB263913.1; AB263914.1; AB263915.1; AB263916.1; AB263917.1; AB263918.1; AB263919.1; AB263920.1; AB263921.1; AB263922.1; AB263923.1; AB263924.1; AB263925.1; AB263926.1; AB263927.1; AB263928.1; AB263929.1; AB263930.1; AB263931.1; AB263932.1; AB263934.1; AB263935.1; AB263936.1; AB263938.1; AB269825.1; AB269826.1; AB269827.1; AB269828.1; AB269829.1; AB269830.1; AB269831.1; AB269832.1; AB269834.1; AB269836.1; AB269837.1; AB269838.1; AB269840.1; AB269841.1; AB269842.1; AB269843.1; AB269844.1; AB269845.1; AB269846.1; AB269847.1; AB269848.1; AB269849.1; AB269850.1; AB269851.1; AB269852.1; AB269853.1; AB269854.1; AB269855.1; AB269856.1; AB269857.1; AB269858.1; AB269859.1; AB269860.1; AB269861.1; AB269862.1; AB269863.1; AB269864.1; AB269865.1; AB269866.1; AB269867.1; AB269868.1; AB269869.1; AB298941.1; AB298942.1; AB298945.1; AB298946.1; AB298947.1; AB301086.1; AB301087.1; AB301089.1; AB301090.1; AB301091.1; AB301092.1; AB301093.1; AB301094.1; AB301095.1; AB301096.1; AB301097.1; AB301099.1; AB301100.1; AB301101.1; AB365130.1; AB365132.1; AB365133.1; AB365134.1; AB365136.1; AB365137.1; AB365138.1; AB365139.1; AB365140.1; AB365141.1; AB365142.1; AB365144.1; AB365145.1; AB365146.1; AB365148.1; AB365149.1; AB365150.1; AB365151.1; AB365153.1; AB365154.1; AB365156.1; AB365157.1; AB365158.1; AB365159.1; AB365160.1; AB365162.1; AB365164.1; AB365165.1; AB365166.1; AB365167.1; AB365168.1; AB365170.1; AB365173.1; AB365174.1; AB365175.1; AB365176.1; AB365178.1; AB369087.1; AB369088.1; AB369089.1; AB369090.1; AB369092.1; AB369093.1; AB369094.1; AB369095.1; AB369096.1; AB369097.1; AB369098.1; AB369099.1; AB369101.1; AB464953.1; AB464954.1; AB464956.1; AB464957.1; AB464958.1; AB464960.1; AB464961.1; AB464962.1; AB485695.1; AB485696.1; AB485697.1; AB485698.1; AB485699.1; AB485700.1; AB485701.1; AB485703.1; AB485704.1; AB485707.1; AB485709.1; AB485710.1; AB485711.1; AB485712.1; AY628224.1; AY628225.1; AY628226.1; AY628227.1; AY628228.1; AY628229.1; AY628230.1; AY628231.1; AY628232.1; AY628233.1; AY628234.1; AY628235.1; AY628236.1; AY628237.1; AY628238.1; DQ305492.1; EF376992.1; FR720308.1; FR720309.1; FR720310.1; FR720311.1; FR720312.1; FR720313.1; FR720315.1; FR720317.1; FR720318.1; FR720320.1; FR720321.1; JF894228.1; JN192431.1; JN192432.1; JN192433.1; JN192435.1; JN192437.1; JN192438.1; JN192439.1; JN192440.1; JQ713822.1; KF055891.1; KF055892.1; KF055893.1; KP412983.1; KP984526.1; KY114802.1; KY114803.1; KY132094.1; KY487998.1; LC029413.1; LC309239.1; LC309240.1; LT960370.1; M23122.1; V01108.1.

Similarly, complete genomic sequences were downloaded for the 13 different prototype human polyomaviruses. The accession numbers are provided below: NC_001538; NC_001699; NC_009238; NC_009539; NC_010277; NC_014406; NC_014407; NC_014361; NC_015150; NC_018102; NC_020106; NC_020890; NC_024118.

Conservation of Large T antigen splice sites Whole genome nucleotide sequences from all reference human polyomaviruses were downloaded from the NCBI website (https://www.ncbi.nlm.nih.gov/nuccore) on Feb. 20, 2018 and aligned with WebPrank (available online: https://www.ebi.ac.uk/goldman-srv/webprank/) using default settings. A phylogenetic UPGMA tree was constructed and sequence logos for every splice site were created to show conservation between different human polyomaviruses. All downloaded refseq accession numbers are provided below: Reference sequences: NC_001538, NC_001699, NC_009238, NC_009539, NC_010277, NC_014406, NC_014407, NC_014361, NC_015150, NC_018102, NC_020106, NC_020890, NC_024118 Whole genome nucleotide sequences for all human polyomavirus isolates were downloaded from the NCBI website on Feb. 20, 2018. Whole gene sequences of Large T antigen were retrieved from only the unique genomic sequences and aligned with WebPrank using default settings Sequence logos were created for every splice site in Large T antigen to show conservation within and between different human polyomaviruses.

All downloaded accession numbers are depicted below:
BKPyV: AB211369.1, AB211370.1, AB211371.1, AB211372.1, AB211373.1, AB211374.1, AB211375.1, AB211376.1, AB211377.1, AB211378.1, AB211379.1, AB211380.1, AB211381.1, AB211382.1, AB211383.1, AB211384.1, AB211385.1, AB211386.1, AB211387.1, AB211388.1, AB211389.1, AB211390.1, AB211391.1, AB213487.1, AB217917.1, AB217918.1, AB217919.1, AB217920.1, AB217921.1, AB260028.1, AB260029.1, AB260030.1, AB260031.1, AB260032.1, AB260033.1, AB260034.1, AB263912.1, AB263913.1, AB263914.1, AB263915.1, AB263916.1, AB263917.1, AB263918.1, AB263919.1, AB263920.1, AB263921.1, AB263922.1, AB263923.1, AB263924.1, AB263925.1, AB263926.1, AB263927.1, AB263928.1, AB263929.1, AB263930.1, AB263931.1, AB263932.1, AB263933.1, AB263934.1, AB263935.1, AB263936.1, AB263937.1, AB263938.1, AB269822.1, AB269823.1, AB269824.1, AB269825.1, AB269826.1, AB269827.1, AB269828.1, AB269829.1, AB269830.1, AB269831.1, AB269832.1, AB269833.1, AB269834.1, AB269835.1, AB269836.1, AB269837.1, AB269838.1, AB269839.1, AB269840.1, AB269841.1, AB269842.1, AB269843.1, AB269844.1, AB269845.1, AB269846.1, AB269847.1, AB269848.1, AB269849.1, AB269850.1, AB269851.1, AB269852.1, AB269853.1, AB269854.1, AB269855.1, AB269856.1, AB269857.1, AB269858.1, AB269859.1, AB269860.1, AB269861.1, AB269862.1, AB269863.1, AB269864.1, AB269865.1, AB269866.1, AB269867.1, AB269868.1, AB269869.1, AB298940.1, AB298941.1, AB298942.1, AB298943.1, AB298944.1, AB298945.1, AB298946.1, AB298947.1, AB301086.1, AB301087.1, AB301088.1, AB301089.1, AB301090.1, AB301091.1, AB301092.1, AB301093.1, AB301094.1, AB301095.1, AB301096.1, AB301097.1, AB301098.1, AB301099.1, AB301100.1, AB301101.1, AB301102.1, AB301103.1, AB365130.1, AB365131.1, AB365132.1, AB365133.1, AB365134.1, AB365135.1, AB365136.1, AB365137.1, AB365138.1, AB365139.1, AB365140.1, AB365141.1, AB365142.1, AB365143.1, AB365144.1, AB365145.1, AB365146.1, AB365147.1, AB365148.1, AB365149.1, AB365150.1, AB365151.1, AB365152.1, AB365153.1, AB365154.1, AB365155.1, AB365156.1, AB365157.1, AB365158.1, AB365159.1, AB365160.1, AB365161.1, AB365162.1, AB365163.1, AB365164.1, AB365165.1, AB365166.1, AB365167.1, AB365168.1, AB365169.1, AB365170.1, AB365171.1, AB365172.1, AB365173.1, AB365174.1, AB365175.1, AB365176.1, AB365177.1, AB365178.1, AB369087.1, AB369088.1, AB369089.1, AB369090.1, AB369091.1, AB369092.1, AB369093.1, AB369094.1, AB369095.1, AB369096.1, AB369097.1, AB369098.1, AB369099.1, AB369100.1, AB369101.1, AB464953.1, AB464954.1, AB464955.1, AB464956.1, AB464957.1, AB464958.1, AB464959.1, AB464960.1, AB464961.1, AB464962.1, AB464963.1, AB485694.1, AB485695.1, AB485696.1, AB485697.1, AB485698.1, AB485699.1, AB485700.1, AB485701.1, AB485702.1, AB485703.1, AB485704.1, AB485705.1, AB485706.1, AB485707.1, AB485708.1, AB485709.1, AB485710.1, AB485711.1, AB485712.1, AY628224.1, AY628225.1, AY628226.1, AY628227.1, AY628228.1, AY628229.1, AY628230.1, AY628231.1, AY628232.1, AY628233.1, AY628234.1, AY628235.1, AY628236.1, AY628237.1, AY628238.1, DQ305492.1, EF376992.1, FR720308.1, FR720309.1, FR720310.1, FR720311.1, FR720312.1, FR720313.1, FR720314.1, FR720315.1, FR720316.1, FR720317.1, FR720318.1, FR720319.1, FR720320.1, FR720321.1, FR720322.1, FR720323.1, JF894228.1, JN192431.1, JN192432.1, JN192433.1, JN192434.1, JN192435.1, JN192436.1, JN192437.1, JN192438.1, JN192439.1, JN192440.1, JN192441.1, JQ713822.1, KF055891.1, KF055892.1, KF055893.1, KP412983.1, KP984526.1, KY114802.1, KY114803.1, KY132094.1, KY487998.1, LC029411.1, LC029412.1, LC029413.1, LC029414.1, LC309239.1, LC309240.1, LT934539.1, LT960370.1, M23122.1, MF627830.1, MF627831.1, V01108.1, V01109.1
Splice Site Conservation and Phylogenetic Trees Whole-gene sequences of large T antigen, including intron sequences, were aligned using clustalW (“msa” package in R) for the 13 different polyomavirus reference sequences and all unique BK-polyomavirus isolates. A phylogenetic tree was constructed using the UPGMA method (“phangorn” and “ggtree” packages in R). A sequence logo was constructed for the acceptor and donor splice sites to show nucleotide specific conservation between subtypes (“msa” package in R).

### ASO Design

Antisense oligonucleotides (ASOs) were designed to target the donor splice site of BK virus large T antigen (SEQ ID NOs: 1 to 10). Additional ASOs were designed derived from SEQ ID NOs: 1 to 10 and with modified chemistry. They contain 2′-O-methyl bases are 20 nucleotides in length and had been further modified with a full phosphorothioate backbone (*) and optionally with 5 methylcytidine where indicated. Secondary structure and binding energy of the ASOs were predicted using RNA structure. All ASO sequences are depicted below:

TABLE 3
Oligonucleotide with modified RNA chemistry

| | | Target splice site |
|---|---|---|
| Name | Sequence | in large T antigen |
| SEQ ID NO: 12 | oC*oC*oU*oC*oU*oG*oA*oG*oC*oU*oA*oC*oU*oC*oC*oA*oG | Donor (exon 1) |
| | *oG*oU*oU | |
| SEQ ID NO: 13 | oA*oA*oC*oC*oU*oC*oU*oG*oA*oG*oC*oU*oA*oC*oU*oC*oC | Donor (exon 1) |
| | *oA*oG*oG | |
| SEQ ID NO: 14 | oC*oA*oA*oA*oC*oC*oU*oC*oU*oG*oA*oG*oC*oU*oA*oC*oU | Donor (exon 1) |
| | *oC*oC*oA | |
| SEQ ID NO: 15 | oC*oA*oC*oA*oA*oA*oC*oC*oU*oC*oU*oG*oA*oG*oC*oU*oA | Donor (exon 1) |
| | *oC*oU*oC | |
| SEQ ID NO: 16 | oA*oG*oC*oA*oC*oA*oA*oA*oC*oC*oU*oC*oU*oG*oA*oG*oC | Donor (exon 1) |
| | *oU*oA*oC | |
| SEQ ID NO: 17 | oU*oC*oA*oG*oC*oA*oC*oA*oA*oA*oC*oC*oU*oC*oU*oG*oA | Donor (exon 1) |
| | *oG*oC*oU | |
| SEQ ID NO: 18 | oA*oA*oU*oC*oA*oG*oC*oA*oC*oA*oA*oA*oC*oC*oU*oC*oU | Donor (exon 1) |
| | *oG*oA*oG | |
| SEQ ID NO: 19 | oA*oA*oA*oA*oU*oC*oA*oG*oC*oA*oC*oA*oA*oA*oC*oC*oU* | Donor (exon 1) |
| | oC*oU*oG | |
| SEQ ID NO: 20 | oG*oG*oA*oA*oA*oA*oU*oC*oA*oG*oC*oA*oC*oA*oA*oA*oC | Donor (exon 1) |
| | *oC*oU*oC | |
| SEQ ID NO: 21 | oG*oA*oG*oG*oA*oA*oA*oA*oU*oC*oA*oG*oC*oA*oC*oA*oA | Donor (exon 1) |
| | *oA*oC*oC | |
| SEQ ID NO: 22 | oA*oG*oC*oA*oC*oA*oA*oA*oC*oC*oU*oC*oU*oG*oA*oG*oC | Donor (exon 1) |
| | *oU*oA | |
| SEQ ID NO: 23 | mA*mG*mC*mA*mC*oA*oA*oA*oC*oC*oU*oC*oU*oG*mA*m | Donor (exon 1) |
| | G*mC*mT*mA | |
| SEQ ID NO: 24 | mA*mG*mC*mA*mC*mA*mA*mA*mC*mC*mT*mC*mT*mG*m | Donor (exon 1) |
| | A*mG*mC*mT*mA | |
| SEQ ID NO: 25 | mA*mG*oC*oA*oC*oA*oA*oA*oC*oC*oU*oC*oU*oG*oA*oG*o | Donor (exon 1) |
| | C*mT*mA | |
| SEQ ID NO: 26 | oA*oG*oC*oA*oC*oA*oA*oA*oC*oC*oU*oC*oU*oG*oA*oG*oC | Donor (exon 1) |
| | *oU*oA | |
| SEQ ID NO: 27 | mT*mC*mA*mG*mC*oA*oC*oA*oA*oA*oC*oC*oU*oC*oU*mG | Donor (exon 1) |
| | *mA*mG*mC*mT | |
| SEQ ID NO: 28 | mT*mC*mA*mG*mC*mA*mC*mA*mA*mA*mC*mC*mT*mC*m | Donor (exon 1) |
| | T*mG*mA*mG*mC*mT | |
| SEQ ID NO: 29 | mT*mC*oA*oG*oC*oA*oC*oA*oA*oA*oC*oC*oU*oC*oU*oG*o | Donor (exon 1) |
| | A*oG*mC*mT | |
| SEQ ID NO: 30 | oU*oC*oA*oG*oC*oA*oC*oA*oA*oA*oC*oC*oU*oC*oU*oG*oA | Donor (exon 1) |
| | *oG*oC*oU | |
| SEQ ID NO: 31 | mA*mA*mT*mC*mA*oG*oC*oA*oC*oA*oA*oA*oC*oC*oU*mC* | Donor (exon 1) |
| | mT*mG*mA*mG | |
| SEQ ID NO: 32 | mA*mA*mT*mC*mA*mG*mC*mA*mC*mA*mA*mA*mC*mC*m | Donor (exon 1) |
| | T*mC*mT*mG*mA*mG | |
| SEQ ID NO: 33 | mA*mA*oU*oC*oA*oG*oC*oA*oC*oA*oA*oA*oC*oC*oU*oC*o | Donor (exon 1) |
| | U*oG*mA*mG | |
| SEQ ID NO: 34 | oA*oA*oU*oC*oA*oG*oC*oA*oC*oA*oA*oA*oC*oC*oU*oC*oU | Donor (exon 1) |
| | *oG*oA*oG | |
| SEQ ID NO: 35 | mA*mA*mA*mA*mT*oC*oA*oG*oC*oA*oC*oA*oA*oA*oC*mC* | Donor (exon 1) |
| | mT*mC*mT*mG | |
| SEQ ID NO: 36 | mA*mA*mA*mA*mT*mC*mA*mG*mC*mA*mC*mA*mA*mA*m | Donor (exon 1) |
| | C*mC*mT*mC*mT*mG | |
| SEQ ID NO: 37 | mA*mA*oA*oA*oU*oC*oA*oG*oC*oA*oC*oA*oA*oA*oC*oC*o | Donor (exon 1) |
| | U*oC*mT*mG | |
| SEQ ID NO: 38 | oA*oA*oA*oA*oU*oC*oA*oG*oC*oA*oC*oA*oA*oA*oC*oC*oU* | Donor (exon 1) |
| | oC*oU*oG | |
| SEQ ID NO: 39 | oA*oA*oA*oA*oU*oC*oA*oG*oC*oU*oC*oA*oA*oA*oC*oC*oU | Donor (exon 1) |
| | *oC*U*oG | |
| SEQ ID NO: 40 | oA*oA*oA*oA*oC*oC*oA*oG*oC*oU*oC*oA*oA*oA*oC*oC*oU* | Donor (exon 1) |
| | oC*oU*oG | |
| SEQ ID NO: 41 | oA*oA*oA*oA*oC*oC*oA*oG*oC*oU*oC*oA*oA*oA*oC*oA*oU* | Donor (exon 1) |
| | oC*oU*oG | |
| SEQ ID NO: 42 | oA*oA*oA*oA*oU*dC*dA*dG*dC*dA*dC*dA*dA*dA*dC*oC*oU* | Donor (exon 1) |
| | oC*oU*oG | |
| SEQ ID NO: 43 | nA*nC*nA*dT*dC*dC*dT*dG*dC*dT*dC*dC*dA*nT*nT*nT | Donor (exon 1) |
| SEQ ID NO: 44 | nG*nG*nT*dG*dA*dA*dA*dT*dT*dC*dC*dT*nT*nA*nC | Donor (exon 1) |
| SEQ ID NO: 45 | nG*nG*nG*dT*dG*dA*dA*dA*dT*dT*dC*dC*dT*nT*nA*nC | Donor (exon 1) |

Note:
*Indicates a phosphorothioate linkage; C indicates 5-methylcytidine; oA, oC, OU, OG and oT indicates 2'-O-methyl modified ribonucleic acid; mA, mC, mU, mG, mT indicates 2'-methoxy ethyl modified ribonucleic acid; dA, dC, dU, dG, dT indicates deoxyribonucleic acid; nA, nC, nU, nG, nT indicates a locked nucleic acid residue. Herein, any modification for C can be applied to C and is represented accordingly.

### Cell Culture

Immortalized proximal tubule kidney epithelial HK2 cells (ATCC® CRL-2190™) were obtained from ATCC and maintained at 37° C., 5% CO₂, in Dulbecco's Modified Eagle's medium-F12, 1:1 mixture with 15 mM Hepes, 2.5 mM L-glutamine (Lonza) and supplemented with Tri-iodo thyronine, epidermal growth factor (EGF), insulin-transferrin-selenium-ethanolamine (ITS-X), hydrocortisone and 100 U/mL penicillin-streptomycin. Human Renal Proximal Tubular Epithelial Cells (PTEpiC) (Sciencell, #4100) were maintained in complete Epithelial Cell Medium (Sciencell, #4101) consisting of 500 ml of basal medium, 2% fetal bovine serum and 1× epithelial cell growth supplement. Experiments with hPTECs were performed between passages 4 and 6. pRPc cells, a mouse cell line transformed with the early coding region of BKV (Negrini, M. et al., Cancer Research, 1992) constitutively express BKV large T-antigen. pRPc cells were maintained in Dulbecco's modified Eagle's medium supplemented with 10% FCS. All cells were cultured at 37° C. and 5% CO₂ in the presence of 100 U/mL penicillin, 100 μg/mL streptomycin solution (Invitrogen, Breda, The Netherlands).

BK polyomavirus (ATCC® VR-837™) was obtained from ATCC and diluted in complete HK2 culture media to reduce the infectious load. For treatment experiments, cells were seeded in 6-well or 12-wells plates (Corning) at a density of 32,000 cells/cm² and grown overnight. ASO treatment was performed by incubating the cells for 5 h with lipofectamine 3000 (Thermo Fisher) at an ASO concentration of 50 nM, after which the lipofectamine was washed off. Infections with BK polyomavirus were performed 24 h after washing of the cells by incubating the cells with BK polyomavirus-containing culture media for 2 h, after which the cells were washed three times to remove excess virus particles. Supernatant was collected after washing these initial washes and at 3, 5 and 7 days after infection to determine the production of viral particles using PCR. A viral load sample was collected before infection to determine the infectious load. RNA and protein were harvested at day 7 to determine the expression of Large T antigen and VP1.

### Viral Load Determinations

In order to determine the viral load in the culture supernatant, 200 μL was collected from every well for every time point. Pierce Universal Nuclease was added to every sample to degrade unpackaged DNA for 15 minutes at RT and was then inactivated with 5 mM EDTA. Viral DNA was isolated from the supernatant using the DNA mini kit (Qiagen) and the viral load was determined using Taqman PCR as described below (Wunderink, H. F., et. al., J. Clin. Virol., 2017).

To monitor the quality of DNA extraction and potential PCR inhibition, we added low concentrations of phocine herpesvirus to the lysis buffer. DNA was eluted in a final volume of 100 μL elution buffer, of which 10 μL was used as input for real-time quantitative PCR (qPCR). Using primers 440BKVs 5′-GAAAAGGAGAGTGTCCAGGG-3′ (SEQ ID NO: 46) and 441 BKVas 5′-GAACTTCTACTCCTCCTTTTATTAGT-3′ (SEQ ID NO: 47) and a Taqman probe 576BKV-TQ-FAM FAM 5′-CCAAAAAGCCAAAGGAACCC-3′-BHQ1, (SEQ ID NO: 48) a 90-bp fragment within the BKPyV VP1 gene was amplified (sequences provided by LUMC Department of Medical Microbiology). The BKPyV qPCR and phocine herpesvirus PCR were duplexed for DNA quality and potential PCR inhibition monitoring. Furthermore, the BKPyV qPCR was validated to detect BKPyV genotypes I-IV.

Quantitative PCR reactions were performed in a total volume of 50 μL, containing 25 μL HotStarTaq Master Mix (QIAGEN, Hilden, Germany), 0.5 μmol/L of each primer, 0.35 μmol/L BKPyV probe, and 3.5 mmol/L MgCl₂. Reactions were performed using a CFX96 real-time detection system (Bio-Rad, Hercules, CA, USA) with the following cycle conditions: 15 min at 95° C. followed by 45 cycles of amplification (30 s at 95° C.; 30 s at 55° C.; 30 s at 72° C.). For quantification, a standard of a quantified BKPyV-positive urine sample was used. Analytical sensitivity of the BKPyV qPCR was ~10 copies/mL. On each plate, 3 negative controls were included; these controls tested negative in all PCR assays. PCR results with a cycle threshold ≥40 were considered negative.

### Antibodies and Western Blotting

Protein concentrations were determined using the BCA method. Samples were run on a 4-15% TGX gel and transferred to a nitrocellulose or PVDF membrane. Antibodies used were: rabbit polyclonal anti-actin-HRP (loading control), rabbit polyclonal anti-SV40 VP1 (ab53977, Abcam), mouse monoclonal anti-SV40 T-antigen [PAb416](ab16879, Abcam), mouse monoclonal anti-SV40 T-Antigen (PAb108, Thermo Fisher), rabbit polyclonal anti-SV40 VP1 (Abcam, ab53977), biotinylated Lotus Tetragonolobus Lectin (LTL; Vector Laboratories, B-1325), sheep polyclonal anti-Nephrin (AF4269, R & D Systems), purified mouse anti-E-cadherin (Becton Dickinson, 610181) rabbit polyclonal anti-GAPDH (Cell Signalling, D16H11) and rabbit polyclonal anti-phosphorothioate (kindly provided by Dr. Jonathan Watts, University of Massachusetts Medical School). Primary antibodies were incubated overnight at 4° C. for Large T antigen and VP1 and 30 minutes at room temperature for actin. Secondary antibodies used for large T antigen and VP1 were goat polyclonal anti-mouse-HRP (P044701-2, Agilent) and goat polyclonal anti-rabbit-HRP (P044801-2, Agilent), respectively. Additionally, goat anti-rabbit 488 (Life Technologies, A-11008), donkey anti-rabbit 488 (Invitrogen, A21206), donkey anti-rat 647 (Invitrogen, ab150155), donkey anti-sheep 647 (Invitrogen, A21448), donkey anti-mouse IgG2a 647 (Invitrogen, A31571), streptavidin 568 (Invitrogen, S11226) and for isotype control rabbit IgG (Dako, X0936). The membranes were incubated with SuperSignal™ West Femto Maximum Sensitivity Substrate (Thermo Fisher) and protein bands were visualized using the ChemiDoc MP Imaging System (Bio Rad). Nuclei were counterstained with Hoechst.

### Immunohistochemistry

Upon sacrificing mice, mouse kidneys, liver, lung, spleen, bladder and heart were excised and perfused with PBS and fixed in 10% buffered formalin and paraffin-embedded. The kidney tissue was sectioned in 4 μm thick sections and mounted on glass slides. Freshly sliced sections were deparaffinized in xylene for 10 minutes after which the sections were rehydrated by in a graded ethanol series and placed in PBS. Subsequently, sections were stained for ASO (using either an antibody that detects the phosphorothioate backbone or by fluorescent in situ hybridization with an RNA-based probe), for kidney injury by staining for kidney injury marker-1 (KIM-1) of the tubuli or interstitial collagen by Sirius red staining.

Real-Time qPCR

ASO-treated and BKV-infected cells were lysed in Trizol and RNA was isolated using the RNeasy kit (Qiagen). A DNAse I (Qiagen) treatment was added to remove excess DNA during the isolation and cDNA was synthesized using Promega reverse transcriptase, DTT, dNTPs and random primers. Real time PCR was performed on a CFX384 Touch™ Real-Time PCR Detection System (Bio Rad) with SYBR™ Select Master Mix (Thermo Fisher) and the following primers:

TABLE 4
Primers

| Gene | Forward | Reverse |
|---|---|---|
| GAPDH | ACAACTTTGGTATCGTGGAAGG | GCCATCACGCCACAGTTTC |
| | (SEQ ID NO: 49) | (SEQ ID NO: 50) |
| TAg | GAGGAGGATGTAAAGGTAGCTCA | ACTGGCAAACATATCTTCA |
| | (SEQ ID NO: 51) | TGGC |
| | | (SEQ ID NO: 52) |
| VP1 | TGCAGGGTCACAAAAAGTGC | AGCACTCCCTGCATTTCCAA |
| | (SEQ ID NO: 53) | (SEQ ID NO: 54) |

Activated Partial Thromboplastin Time (aPTT) Assay

A magnetic ball was added to a StartMax cuvette (Diagnostica Stago). Plasma was diluted with Owren-Kollerdiluent (Diagnostica Stago) after which 50 μL aPTT reagent (TriniClot) was added percuvette. Subsequently, 50 μL diluted plasma containing 5 uL ASO solution was added to the aPTT reagent and incubated for 170 seconds at 37° C. Next, the cuvette was placed in the magnetic field and the magnetic field activated to initiate coagulation. At 180 seconds 50 μL 25 mM CaCl₂) was added per cuvette with the repeater pipette, and coagulation time measurements automatically recorded with every pipette action.

### Peripheral Blood Mononuclear Cell Cytokine Production Assay

PBMCs were thawed and added to culture medium for 5 minutes and collected by centrifugation. The cells were resuspended and initial viability assessed by trypan blue exclusion, after which the cells were diluted to 1.67×10⁶ cells/mL. Subsequently, negative controls (culture medium) and positive controls were prepared in culture medium (R848 (Invivogen, USA)). Cytokine production in response to exposure to 1 μM or 10 μM ASO was assessed by adding 20 μL negative or positive control or appropriate ASO concentration to a round-bottom well after which 180 μL culture medium containing PBMCs was added and incubated at 37° C. (5% CO₂) for 48 h. Subsequently, the culture medium was pipetted to Eppendorf tubes, centrifuged at 1200 rpm for 6 minutes and supernatant transferred to custom multiplex elisa plates allowing for the detection of GM-CSF, IFN-gamma, IL-6, IL-12 (p70 subunit), MIP-1b, TNF-alpha, G-CSF, IFN-alpha 2, IL-1b, IL-2, IL-10 and IL-17.

Cell viability was assessed by adding negative or positive control material or ASO in flat-bottom wells and 190 μL culture medium containing PBMCs and incubated for 48 h at 37° C. (5% CO₂). Subsequently, CellTiter-Blue reagent was added per well, the wells were mixed for 4 h and incubated for 4 h after which fluorescence was measured at 555/585 nm.

Hybridization ELISA (hELISA)

Tissue harvested from mice was placed in lysis buffer and diluted to a non-saturating concentration. Standard curves and tissue samples were subsequently diluted 1:50 in sample buffer by adding 1 μL of standard curve or tissue sample to 49 μL sample buffer in a single well of a 96-well plate. Next, 50 μL probe mixture was added per well (consisting of 20 nM capture probe and 20 nM detection probe) after which the 96-well plate was covered with a light-refracting seal. Next, the probes were hybridized in a thermal cycler at 95° C. for 5 minutes, 40° C. for 30 minutes and a final hold at 12° C. Next, MSD Gold plates (Mesoscale) were prepared by washing with KPL wash buffer after which the hybridized samples were transferred from the 96-well plates to the MSD Gold plates in duplicate wells. The MSD Gold plates were covered with a light-refracting seal and incubated at RT at 650 rpm on an orbital shaker for 30 minutes. Next, each well was washed 3× with KPL buffer after which 0.5 μg/mL SULFO-tag anti-digoxigenin antibody (in 1% blocker A buffer) was added to each well. The plate was sealed with light-refracting strip and incubated for 60 minutes on an orbital shaker at 650 rpm. The plates were subsequently washed 3× with KPL buffer after which MSD Gold buffer was added to each well and the plate read by spectrophotometry.

### Animals

C57B16 wild-type mice were housed at the Leiden University Medical Center animal facility. Mice received chow diet and water ad libitum. For biodistribution and preliminary safety studies, ASO was administered intravenously via the tail vein (40 mg/kg) on days 0, 3, 7 and 10. Upon sacrifice (day 14), blood (and urine) was collected from each mouse and allowed to stand at room temperature for 30 minutes, then centrifuged at 6,000 rpm at 4° C. to collect the upper serum. Creatinine, blood urea nitrogen (BUN or urea), albumin, aspartate aminotransferase (AST) and alanine aminotransferase (ALT) were determined at the Clinical Chemistry Laboratory at the LUMC.

### Example 2

### Results

ASOs According to the Invention have Improved Antiviral Activity

In order to exemplify antiviral activity of ASOs according to the invention, the antiviral activity of the ASO according to SEQ ID NO: 8 (ASO8) was compared to the antiviral activity of the structurally similar ASOs according to SEQ ID NOs 23 and 24 (ASO23 and ASO24), respectively, of WO2019/168402. ASO23 and ASO24 differ from ASO8 only in that their 20 nucleotide target region in the polyomavirus large T-antigen pre-mRNA starts one nucleotide upstream or downstream, respectively

Human Renal Proximal Epithelial cells (PTEC) were treated with 50 nM ASO 24 h prior to infection with BKV, and BKV RNA and protein expression were quantified at day 5 post-infection. In this in vitro model, treatment with ASO8 resulted in a reduction of T-Ag and VP1 mRNA of 93.8% and 96.5% respectively relative to an untreated control. These reductions were superior to reductions achieved with ASO23 (T-Ag: 91.9% and VP1: 92.0%) and ASO24 (T-Ag: 92.4% and VP1: 90.4%). See FIG. 17A. The improvement afforded by ASO8 was confirmed by the determination of downstream VP1 protein expression, which was inhibited more effectively following treatment with ASO8 (92.5%) compared to ASO23 and ASO24 at 80.7% and 89.4% respectively. See FIG. 17B. Overall, these results demonstrate that ASO8 was efficient at inhibiting BKV replication, and improved compared to structurally similar ASOs. This improvement was entirely unexpected in view of the structural similarities and, in consequence, the almost identical target region.

### Material and Methods

### Transfection and Infection of Cells

Human Renal Proximal Epithelial cells (PTECS, Sciencell, Cat: 4100) were maintained at 37° C. and 5% CO₂ in REBM Basal medium (Lonza, Cat: CC-3191) with REGM™ SingleQuots™ supplements (Lonza, Cat: CC-4127, from here on called REGM) supplemented to 0.5% FCS. Before transfection, PTEC were seeded overnight in 12 wells plates (Corning, Cat: 3512) at a density of 21,000 cells/cm². Transfection of 50 nM ASO was achieved using Lipofectamine 3000 (Invitrogen, Cat: L3000075) following manufacturer's instructions. After 5 h incubation, all REGM was replaced with fresh REGM and cells were maintained overnight, after which cells were infected for 2 h with BKV (ATCC, Cat: VR-837; diluted 1:3000 from original stock). The virus-containing REGM was aspirated and all wells were washed 3× before fresh REGM was added. Cells were maintained in culture for 5 days post-infection, with supplementation of REGM at day 3 post-infection. Untreated cells were taken along as control.

### Quantitative Real Time PCR

RNA was isolated using the RNeasy kit (Qiagen, Cat: 74106) following manufacturer's instructions with RLT buffer and 1% 2-Mercaptoethanol (Sigma-Aldrich, Cat: M3148-100 mL) and DNAse I treatment (Qiagen, Cat: 1010395). An additional DNA removal step was included after RNA elution to remove any excess BKV DNA using the TURBO DNA-Free™ Kit (Thermo Fisher Scientific, Cat: AM1907). Synthesis of cDNA was performed using M-MLV Reverse Transcriptase (Cat: M1708) in M-MLV RT 5× buffer (Cat: M531A), 10 mM dNTP Mix (Cat: U1518), RNasin® Ribonuclease Inhibitor (Cat: N2518), Oligo(dT)15 Primer (Cat: C110A) and 0.1M Molecular Grade DTT (all Promega, Cat: Y00147). Quantitative real-time PCR analysis of BKV RNA expression was performed on a CFX Opus Real-Time PCR System (Bio-Rad) according to the table below using SYBR Select Master Mix (Thermo Fisher Scientific, Cat: 4472908) with the following primers, T-Ag: GAGGAGGATGTAAAGGTAGCTCA (forward, SEQ ID NO: 70) and ACTGGCAAACATATCTTCATGGC (reverse, SEQ ID NO: 71); VP1: TGCAGGGTCACAAAAAGTGC (forward, SEQ ID NO: 72) and AGCACTCCCTGCATTTCCAA (reverse, SEQ ID NO: 73); GAPDH: ACAACTTTGGTATCGTGGAAGG (forward, SEQ ID NO: 74) and GCCATCACGCCACAGTTTC (reverse, SEQ ID NO: 75). Changes in mRNA expression were determined using the ΔΔCt method.

| Step | °C | Time | | |
|---|---|---|---|---|
| 1 | 50 | 2 | min | |
| 2 | 95 | 2 | min | |
| 3 | 95 | 10 | sec | 39x |
| 4 | 61 | 20 | sec | |
| 5 | 72 | 45 | sec | |
| 6 | 95 | 10 | Sec | Increment 0.5 + Plate read |

| | | |
|---|---|---|
| 7 | 65-95 | Melting curves |
| 8 | 4 | Hold |

### VP1 Protein Quantification Using Simple Western Analysis

Cells were lysed at 5 d post-infection in RIPA Lysis and Extraction Buffer (ThermoFisher Scientific, Cat: 89901) with 1:100 Pierce Protease and Phosphatase Inhibitor Mini Tablets (ThermoFisher Scientific, Cat: A32959) and protein concentrations were determined using the Pierce™ BCA Protein Assay Kit (Thermo Scientific, Cat: 23225). VP1 protein expression was quantified using the Jess Simple Western System (Biotechne). In short, 0.5 mg/mL protein lysate samples were analyzed using a 12-230 kDa Separation Module (ProteinSimple, Cat: SM-W004-1) and Anti-Rabbit Detection Module (ProteinSimple, Cat: DM-001). When required, antibodies were diluted in Antibody Diluent. VP1 protein was detected using 1:20 Anti-SV40 VP1 antibody (Abcam, Cat: ab53977) and 1:20 Goat Anti-Rabbit Immunoglobulins/HRP (Agilent, Cat: P044801-2). Beta-Actin protein expression was used as loading control using 1:20 β-Actin Mouse monoclonal Antibody (Cell Signaling Technology, Cat: #3700S) with 1:20 Goat Anti-Mouse Immunoglobulins/HRP (Agilent, Cat: P044701-2) used as secondary antibody. The assay consisted of a 30 minutes separation time at a voltage of 375V, followed by 5 min antibody diluent time, 60 minutes primary antibody time and 30 minutes secondary antibody time. Peak area calculation was performed using dropped lines of the High Dynamic Range chemiluminescent signal. Peak find threshold and width were adjusted on a capillary-by-capillary basis to ensure proper fitting of the signal.

1. An oligonucleotide comprising a nucleobase sequence according to one of SEQ ID NOs: 8, 1, 2, 3, 4, 5, 6, 7, 9, 10 and 11 or comprising a nucleobase sequence which is analogous to any one of SEQ ID NOs: 1 to 11 characterized in that at least one nucleobase of said SEQ ID NOs is replaced by a nucleobase analogue having the same base pairing specificity as the replaced nucleobase.

2. The oligonucleotide according to claim 1, wherein the nucleobase sequence of the oligonucleotide consists of the nucleobase sequence according to one of SEQ ID NOs: 1 to 11, or wherein the nucleobase sequence of the oligonucleotide consists of a nucleobase sequence that is analogous to any one of SEQ ID NOs: 1 to 11 characterized in that at least one nucleobase of said SEQ ID NOs is replaced by a nucleobase analogue having the same base pairing specificity as the replaced nucleobase.

3. The oligonucleotide according to claim 1, wherein the oligonucleotide comprises a modification capable of rendering an RNA duplex resistant to RNase H, wherein the RNA duplex comprises the oligonucleotide and an oligonucleotide complementary thereto (complementary oligonucleotide).

4. The oligonucleotide according to claim 1, wherein the oligonucleotide comprises a modified internucleotide linkage, preferably a phosphorothioate internucleotide linkage.

5. The oligonucleotide according to claim 4, wherein the oligonucleotide comprises a nucleotide sequence according to one of SEQ ID NOs: 12 to 21, or comprises a nucleotide sequence according SEQ ID NO: 22, preferably wherein the nucleotide sequence of the oligonucleotide consists of a nucleotide sequence according one of SEQ ID NOs: 12 to 22.

6. The oligonucleotide according to claim 5, wherein the oligonucleotide comprises a nucleotide sequence according to SEQ ID NO: 19, preferably wherein the nucleotide sequence of the oligonucleotide consists of a nucleotide sequence according to SEQ ID NO: 19.

7. The oligonucleotide according to claim 4, comprising unmodified internucleotide linkages between nucleotides 5 to 16 of SEQ ID NOs: 1 to 11 and modified internucleotide linkages between at least the two most 5′ end nucleotides of the oligonucleotide and between at least the two most 3′ end nucleotides of the oligonucleotide.

8. A vector, preferably a viral vector, comprising (i) an oligonucleotide as defined in claim 1, (ii) the reverse complement of an oligonucleotide as defined in claim 1, or (iii) DNA capable of being transcribed to an oligonucleotide as defined in claim 1.

9. A pharmaceutical composition comprising an oligonucleotide as defined in claim 1.

10. A method of treating a subject, comprising administering the oligonucleotide as defined in a claim 1.

11. A method of treating a polyomavirus infection in a subject, comprising administering the oligonucleotide as defined in a claim 1.

12. The method of claim 10, wherein the oligonucleotide is administered to an immunocompromised subject.

13. The method of claim 12, wherein the immunocompromised subject is a transplant recipient, preferably a kidney transplant recipient.

14. An ex-vivo method of inhibiting polyomavirus replication in a cell, the method comprising
(i) providing a cell that infected with polyomavirus, and contacting the cell with an oligonucleotide as defined in claim 1; or
(ii) providing a cell comprising an oligonucleotide as defined in claim 1, and contacting the cell with polyomavirus.

15. An ex-vivo method of producing a transplant, the method comprising providing a donor organ, tissue or cell(s), preferably comprising kidney cells, and contacting cells of the donor organ, tissue or cell(s) with an oligonucleotide as defined in claim 1.

16. The method of claim 10, wherein the oligonucleotide is capable of exhibiting at least one of the following effects:
1) Modulating the splicing of T-antigen pre-mRNA,
2) Reducing the production of T-antigen mRNA,
3) Reducing the production of VP1 mRNA and preferably VP1 protein,
4) Inhibiting virus replication,
5) Limiting the capacity of virus reinfection.
6) Preventing a cellular effect associated with an infection,
7) Alleviating a cellular effect associated with the infection,
8) Preventing disease in an infected subject, and
9) Slowing down, preferably stopping, more preferably reversing disease progression.

17. The method of claim 16,
wherein the disease has been prevented (8) or progression slowed down, stopped or reversed (9) when at least one parameters of the disease associated with said polyomavirus infection has been reduced, said parameters being selected from: glomerular filtration rate, creatinine levels, cyclin E2 (CCNE2), cell division cycle 6 (CDC6), cyclin E2 (CCNA2), E2F transcription factor 8 (E2F8), survivin (BIRC5), RAD51-associated protein-1 (RAD51AP1), BRCA1-interacting protein C-terminal helicase 1 (BRIP1) and apolipoprotein B mRNA-editing enzyme 3B (APOBEC3B).


SEQUENCE LISTING (77 sequences)

SEQ ID NO: 1 - RNA, 20 nt, synthetic construct
  misc_feature 1..20: note = antisense nucleic acid molecule
  source 1..20: mol_type = other RNA
cctctgagctactccaggtt

SEQ ID NO: 2 - RNA, 20 nt, synthetic construct
  misc_feature 1..20: note = antisense nucleic acid molecule
  source 1..20: mol_type = other RNA
aacctctgagctactccagg

SEQ ID NO: 3 - RNA, 20 nt, synthetic construct
  misc_feature 1..20: note = antisense nucleic acid molecule
  source 1..20: mol_type = other RNA
caaacctctgagctactcca

SEQ ID NO: 4 - RNA, 20 nt, synthetic construct
  misc_feature 1..20: note = antisense nucleic acid molecule
  source 1..20: mol_type = other RNA
cacaaacctctgagctactc

SEQ ID NO: 5 - RNA, 20 nt, synthetic construct
  misc_feature 1..20: note = antisense nucleic acid molecule
  source 1..20: mol_type = other RNA
agcacaaacctctgagctac

SEQ ID NO: 6 - RNA, 20 nt, synthetic construct
  misc_feature 1..20: note = antisense nucleic acid molecule
  source 1..20: mol_type = other RNA
tcagcacaaacctctgagct

SEQ ID NO: 7 - RNA, 20 nt, synthetic construct
  misc_feature 1..20: note = antisense nucleic acid molecule
  source 1..20: mol_type = other RNA
aatcagcacaaacctctgag

SEQ ID NO: 8 - RNA, 20 nt, synthetic construct
  misc_feature 1..20: note = antisense nucleic acid molecule
  source 1..20: mol_type = other RNA
aaaatcagcacaaacctctg

SEQ ID NO: 9 - RNA, 20 nt, synthetic construct
  misc_feature 1..20: note = antisense nucleic acid molecule
  source 1..20: mol_type = other RNA
ggaaaatcagcacaaacctc

SEQ ID NO: 10 - RNA, 20 nt, synthetic construct
  misc_feature 1..20: note = antisense nucleic acid molecule
  source 1..20: mol_type = other RNA
gaggaaaatcagcacaaacc

SEQ ID NO: 11 - RNA, 19 nt, synthetic construct
  misc_feature 1..19: note = antisense nucleic acid molecule
  source 1..19: mol_type = other RNA
agcacaaacctctgagcta

SEQ ID NO: 12 - RNA, 20 nt, synthetic construct
  misc_feature 1..20: note = antisense nucleic acid molecule
  modified_base 1..19: mod_base = OTHER; note = phosphorothioate linkage
  modified_base 1..20: mod_base = OTHER; note = 2'-O-methyl
  source 1..20: mol_type = other RNA
cctctgagctactccaggtt

SEQ ID NO: 13 - RNA, 20 nt, synthetic construct
  misc_feature 1..20: note = antisense nucleic acid molecule
  modified_base 1..19: mod_base = OTHER; note = phosphorothioate linkage
  modified_base 1..20: mod_base = OTHER; note = 2'-O-methyl
  source 1..20: mol_type = other RNA
aacctctgagctactccagg

SEQ ID NO: 14 - RNA, 20 nt, synthetic construct
  misc_feature 1..20: note = antisense nucleic acid molecule
  modified_base 1..19: mod_base = OTHER; note = phosphorothioate linkage
  modified_base 1..20: mod_base = OTHER; note = 2'-O-methyl
  source 1..20: mol_type = other RNA
caaacctctgagctactcca

SEQ ID NO: 15 - RNA, 20 nt, synthetic construct
  misc_feature 1..20: note = antisense nucleic acid molecule
  modified_base 1..19: mod_base = OTHER; note = phosphorothioate linkage
  modified_base 1..20: mod_base = OTHER; note = 2'-O-methyl
  source 1..20: mol_type = other RNA
cacaaacctctgagctactc

SEQ ID NO: 16 - RNA, 20 nt, synthetic construct
  misc_feature 1..20: note = antisense nucleic acid molecule
  modified_base 1..19: mod_base = OTHER; note = phosphorothioate linkage
  modified_base 1..20: mod_base = OTHER; note = 2'-O-methyl
  source 1..20: mol_type = other RNA
agcacaaacctctgagctac

SEQ ID NO: 17 - RNA, 20 nt, synthetic construct
  misc_feature 1..20: note = antisense nucleic acid molecule
  modified_base 1..19: mod_base = OTHER; note = phosphorothioate linkage
  modified_base 1..20: mod_base = OTHER; note = 2'-O-methyl
  source 1..20: mol_type = other RNA
tcagcacaaacctctgagct

SEQ ID NO: 18 - RNA, 20 nt, synthetic construct
  misc_feature 1..20: note = antisense nucleic acid molecule
  modified_base 1..19: mod_base = OTHER; note = phosphorothioate linkage
  modified_base 1..20: mod_base = OTHER; note = 2'-O-methyl
  source 1..20: mol_type = other RNA
aatcagcacaaacctctgag

SEQ ID NO: 19 - RNA, 20 nt, synthetic construct
  misc_feature 1..20: note = antisense nucleic acid molecule
  modified_base 1..19: mod_base = OTHER; note = phosphorothioate linkage
  modified_base 1..20: mod_base = OTHER; note = 2'-O-methyl
  source 1..20: mol_type = other RNA
aaaatcagcacaaacctctg

SEQ ID NO: 20 - RNA, 20 nt, synthetic construct
  misc_feature 1..20: note = antisense nucleic acid molecule
  modified_base 1..19: mod_base = OTHER; note = phosphorothioate linkage
  modified_base 1..20: mod_base = OTHER; note = 2'-O-methyl
  source 1..20: mol_type = other RNA
ggaaaatcagcacaaacctc

SEQ ID NO: 21 - RNA, 20 nt, synthetic construct
  misc_feature 1..20: note = antisense nucleic acid molecule
  modified_base 1..19: mod_base = OTHER; note = phosphorothioate linkage
  modified_base 1..20: mod_base = OTHER; note = 2'-O-methyl
  source 1..20: mol_type = other RNA
gaggaaaatcagcacaaacc

SEQ ID NO: 22 - RNA, 19 nt, synthetic construct
  misc_feature 1..19: note = antisense nucleic acid molecule
  modified_base 1..18: mod_base = OTHER; note = phosphorothioate linkage
  modified_base 1..19: mod_base = OTHER; note = 2'-O-methyl
  source 1..19: mol_type = other RNA
agcacaaacctctgagcta

SEQ ID NO: 23 - RNA, 19 nt, synthetic construct
  modified_base 18: mod_base = OTHER; note = thymine
  misc_feature 1..19: note = antisense nucleic acid molecule
  modified_base 1..18: mod_base = OTHER; note = phosphorothioate linkage
  modified_base 1..5: mod_base = OTHER; note = 2'-methoxy-ethyl
  modified_base 15..19: mod_base = OTHER; note = 2'-methoxy-ethyl
  modified_base 6..14: mod_base = OTHER; note = 2'-O-methyl
  source 1..19: mol_type = other RNA
agcacaaacctctgagcta

SEQ ID NO: 24 - RNA, 19 nt, synthetic construct
  modified_base 11: mod_base = OTHER; note = thymine
  modified_base 13: mod_base = OTHER; note = thymine
  modified_base 18: mod_base = OTHER; note = thymine
  misc_feature 1..19: note = antisense nucleic acid molecule
  modified_base 1..18: mod_base = OTHER; note = phosphorothioate linkage
  modified_base 1..19: mod_base = OTHER; note = 2'-methoxy-ethyl
  source 1..19: mol_type = other RNA
agcacaaacctctgagcta

SEQ ID NO: 25 - RNA, 19 nt, synthetic construct
  modified_base 18: mod_base = OTHER; note = thymine
  misc_feature 1..19: note = antisense nucleic acid molecule
  modified_base 1..18: mod_base = OTHER; note = phosphorothioate linkage
  modified_base 1..2: mod_base = OTHER; note = 2'-methoxy-ethyl
  modified_base 18..19: mod_base = OTHER; note = 2'-methoxy-ethyl
  ncRNA 3..17: ncRNA_class = other; note = 2'-O-methyl
  source 1..19: mol_type = other RNA
agcacaaacctctgagcta

SEQ ID NO: 26 - RNA, 19 nt, synthetic construct
  misc_feature 1..19: note = antisense nucleic acid molecule
  modified_base 1..18: mod_base = OTHER; note = phosphorothioate linkage
  modified_base 1..19: mod_base = OTHER; note = 2'-O-methyl
  modified_base 3: mod_base = m5c
  modified_base 5: mod_base = m5c
  modified_base 9..10: mod_base = m5c
  modified_base 12: mod_base = m5c
  modified_base 17: mod_base = m5c
  source 1..19: mol_type = other RNA
agcacaaacctctgagcta

SEQ ID NO: 27 - RNA, 20 nt, synthetic construct
  modified_base 1: mod_base = OTHER; note = thymine
  modified_base 20: mod_base = OTHER; note = thymine
  misc_feature 1..20: note = antisense nucleic acid molecule
  modified_base 1..19: mod_base = OTHER; note = phosphorothioate linkage
  modified_base 1..5: mod_base = OTHER; note = 2'-methoxy-ethyl
  modified_base 16..20: mod_base = OTHER; note = 2'-methoxy-ethyl
  modified_base 6..15: mod_base = OTHER; note = 2'-O-methyl
  source 1..20: mol_type = other RNA
tcagcacaaacctctgagct

SEQ ID NO: 28 - RNA, 20 nt, synthetic construct
  modified_base 1: mod_base = OTHER; note = thymine
  modified_base 13: mod_base = OTHER; note = thymine
  modified_base 15: mod_base = OTHER; note = thymine
  modified_base 20: mod_base = OTHER; note = thymine
  misc_feature 1..20: note = antisense nucleic acid molecule
  modified_base 1..19: mod_base = OTHER; note = phosphorothioate linkage
  modified_base 1..20: mod_base = OTHER; note = 2'-methoxy-ethyl
  source 1..20: mol_type = other RNA
tcagcacaaacctctgagct

SEQ ID NO: 29 - RNA, 20 nt, synthetic construct
  modified_base 1: mod_base = OTHER; note = thymine
  modified_base 20: mod_base = OTHER; note = thymine
  misc_feature 1..20: note = antisense nucleic acid molecule
  modified_base 1..19: mod_base = OTHER; note = phosphorothioate linkage
  modified_base 1..2: mod_base = OTHER; note = 2'-methoxy-ethyl
  modified_base 19..20: mod_base = OTHER; note = 2'-methoxy-ethyl
  modified_base 3..18: mod_base = OTHER; note = 2'-O-methyl
  source 1..20: mol_type = other RNA
tcagcacaaacctctgagct

SEQ ID NO: 30 - RNA, 20 nt, synthetic construct
  misc_feature 1..20: note = antisense nucleic acid molecule
  modified_base 1..19: mod_base = OTHER; note = phosphorothioate linkage
  modified_base 1..20: mod_base = OTHER; note = 2'-O-methyl
  modified_base 2: mod_base = m5c
  modified_base 5: mod_base = m5c
  modified_base 7: mod_base = m5c
  modified_base 11..12: mod_base = m5c
  modified_base 14: mod_base = m5c
  modified_base 19: mod_base = m5c
  source 1..20: mol_type = other RNA
tcagcacaaacctctgagct

SEQ ID NO: 31 - RNA, 20 nt, synthetic construct
  modified_base 3: mod_base = OTHER; note = thymine
  modified_base 17: mod_base = OTHER; note = thymine
  misc_feature 1..20: note = antisense nucleic acid molecule
  modified_base 1..19: mod_base = OTHER; note = phosphorothioate linkage
  modified_base 1..5: mod_base = OTHER; note = 2'-methoxy-ethyl
  modified_base 16..20: mod_base = OTHER; note = 2'-methoxy-ethyl
  modified_base 6..15: mod_base = OTHER; note = 2'-O-methyl
  source 1..20: mol_type = other RNA
aatcagcacaaacctctgag

SEQ ID NO: 32 - RNA, 20 nt, synthetic construct
  modified_base 3: mod_base = OTHER; note = thymine
  modified_base 15: mod_base = OTHER; note = thymine
  modified_base 17: mod_base = OTHER; note = thymine
  misc_feature 1..20: note = antisense nucleic acid molecule
  modified_base 1..19: mod_base = OTHER; note = phosphorothioate linkage
  modified_base 1..20: mod_base = OTHER; note = 2'-methoxy-ethyl
  source 1..20: mol_type = other RNA
aatcagcacaaacctctgag

SEQ ID NO: 33 - RNA, 20 nt, synthetic construct
  misc_feature 1..20: note = antisense nucleic acid molecule
  modified_base 1..19: mod_base = OTHER; note = phosphorothioate linkage
  modified_base 1..2: mod_base = OTHER; note = 2'-methoxy-ethyl
  modified_base 19..20: mod_base = OTHER; note = 2'-methoxy-ethyl
  modified_base 3..18: mod_base = OTHER; note = 2'-O-methyl
  source 1..20: mol_type = other RNA
aatcagcacaaacctctgag

SEQ ID NO: 34 - RNA, 20 nt, synthetic construct
  misc_feature 1..20: note = antisense nucleic acid molecule
  modified_base 1..19: mod_base = OTHER; note = phosphorothioate linkage
  modified_base 1..20: mod_base = OTHER; note = 2'-O-methyl
  modified_base 4: mod_base = m5c
  modified_base 7: mod_base = m5c
  modified_base 9: mod_base = m5c
  modified_base 13..14: mod_base = m5c
  modified_base 16: mod_base = m5c
  source 1..20: mol_type = other RNA
aatcagcacaaacctctgag

SEQ ID NO: 35 - RNA, 20 nt, synthetic construct
  modified_base 5: mod_base = OTHER; note = thymine
  modified_base 17: mod_base = OTHER; note = thymine
  modified_base 19: mod_base = OTHER; note = thymine
  misc_feature 1..20: note = antisense nucleic acid molecule
  modified_base 1..19: mod_base = OTHER; note = phosphorothioate linkage
  modified_base 1..5: mod_base = OTHER; note = 2'-methoxy-ethyl
  modified_base 16..20: mod_base = OTHER; note = 2'-methoxy-ethyl
  modified_base 6..15: mod_base = OTHER; note = 2'-O-methyl
  source 1..20: mol_type = other RNA
aaaatcagcacaaacctctg

SEQ ID NO: 36 - RNA, 20 nt, synthetic construct
  modified_base 5: mod_base = OTHER; note = thymine
  modified_base 17: mod_base = OTHER; note = thymine
  modified_base 19: mod_base = OTHER; note = thymine
  misc_feature 1..20: note = antisense nucleic acid molecule
  modified_base 1..19: mod_base = OTHER; note = phosphorothioate linkage
  modified_base 1..20: mod_base = OTHER; note = 2'-methoxy-ethyl
  source 1..20: mol_type = other RNA
aaaatcagcacaaacctctg

SEQ ID NO: 37 - RNA, 20 nt, synthetic construct
  modified_base 19: mod_base = OTHER; note = thymine
  misc_feature 1..20: note = antisense nucleic acid molecule
  modified_base 1..19: mod_base = OTHER; note = phosphorothioate linkage
  modified_base 1..2: mod_base = OTHER; note = 2'-methoxy-ethyl
  modified_base 19..20: mod_base = OTHER; note = 2'-methoxy-ethyl
  modified_base 3..18: mod_base = OTHER; note = 2'-O-methyl
  source 1..20: mol_type = other RNA
aaaatcagcacaaacctctg

SEQ ID NO: 38 - RNA, 20 nt, synthetic construct
  misc_feature 1..20: note = antisense nucleic acid molecule
  modified_base 1..19: mod_base = OTHER; note = phosphorothioate linkage
  modified_base 1..20: mod_base = OTHER; note = 2'-O-methyl
  modified_base 6: mod_base = m5c
  modified_base 9: mod_base = m5c
  modified_base 11: mod_base = m5c
  modified_base 15..16: mod_base = m5c
  modified_base 18: mod_base = m5c
  source 1..20: mol_type = other RNA
aaaatcagcacaaacctctg

SEQ ID NO: 39 - RNA, 20 nt, synthetic construct
  misc_feature 1..20: note = antisense nucleic acid molecule
  modified_base 1..19: mod_base = OTHER; note = phosphorothioate linkage
  modified_base 1..20: mod_base = OTHER; note = 2'-O-methyl
  source 1..20: mol_type = other RNA
aaaatcagctcaaacctctg

SEQ ID NO: 40 - RNA, 20 nt, synthetic construct
  misc_feature 1..20: note = antisense nucleic acid molecule
  modified_base 1..19: mod_base = OTHER; note = phosphorothioate linkage
  modified_base 1..20: mod_base = OTHER; note = 2'-O-methyl
  source 1..20: mol_type = other RNA
aaaaccagctcaaacctctg

SEQ ID NO: 41 - RNA, 20 nt, synthetic construct
  misc_feature 1..20: note = antisense nucleic acid molecule
  modified_base 1..19: mod_base = OTHER; note = phosphorothioate linkage
  modified_base 1..20: mod_base = OTHER; note = 2'-O-methyl
  source 1..20: mol_type = other RNA
aaaaccagctcaaacatctg

SEQ ID NO: 42 - RNA, 20 nt, synthetic construct
  misc_feature 1..20: note = antisense nucleic acid molecule
  modified_base 1..19: mod_base = OTHER; note = phosphorothioate linkage
  modified_base 1..5: mod_base = OTHER; note = 2'-O-methyl
  modified_base 16..20: mod_base = OTHER; note = 2'-O-methyl
  misc_feature 6..15: note = deoxyribonucleic acid
  source 1..20: mol_type = other RNA
aaaatcagcacaaacctctg

SEQ ID NO: 43 - RNA, 16 nt, synthetic construct
  modified_base 4: mod_base = OTHER; note = thymine
  modified_base 7: mod_base = OTHER; note = thymine
  modified_base 10: mod_base = OTHER; note = thymine
  modified_base 14..16: mod_base = OTHER; note = thymine
  misc_feature 1..16: note = antisense nucleic acid molecule
  modified_base 1..15: mod_base = OTHER; note = phosphorothioate linkage
  modified_base 1..3: mod_base = OTHER; note = locked nucleic acid
  modified_base 14..16: mod_base = OTHER; note = locked nucleic acid
  misc_feature 4..13: note = deoxyribonucleic acid
  source 1..16: mol_type = other RNA
acatcctgctccattt

SEQ ID NO: 44 - RNA, 15 nt, synthetic construct
  modified_base 3: mod_base = OTHER; note = thymine
  modified_base 8..9: mod_base = OTHER; note = thymine
  modified_base 12..13: mod_base = OTHER; note = thymine
  misc_feature 1..15: note = antisense nucleic acid molecule
  modified_base 1..14: mod_base = OTHER; note = phosphorothioate linkage
  modified_base 1..3: mod_base = OTHER; note = locked nucleic acid
  modified_base 13..15: mod_base = OTHER; note = locked nucleic acid
  misc_feature 4..12: note = deoxyribonucleic acid
  source 1..15: mol_type = other RNA
ggtgaaattccttac

SEQ ID NO: 45 - RNA, 16 nt, synthetic construct
  modified_base 4: mod_base = OTHER; note = thymine
  modified_base 9..10: mod_base = OTHER; note = thymine
  modified_base 13..14: mod_base = OTHER; note = thymine
  misc_feature 1..16: note = antisense nucleic acid molecule
  modified_base 1..15: mod_base = OTHER; note = phosphorothioate linkage
  modified_base 1..3: mod_base = OTHER; note = locked nucleic acid
  modified_base 14..16: mod_base = OTHER; note = locked nucleic acid
  misc_feature 4..13: note = deoxyribonucleic acid
  source 1..16: mol_type = other RNA
gggtgaaattccttac

SEQ ID NO: 46 - DNA, 20 bp, synthetic construct
  misc_feature 1..20: note = 440BKVs primer
  source 1..20: mol_type = other DNA
gaaaaggagagtgtccaggg

SEQ ID NO: 47 - DNA, 26 bp, synthetic construct
  misc_feature 1..26: note = 441BKVas primer
  source 1..26: mol_type = other DNA
gaacttctactcctccttttattagt

SEQ ID NO: 48 - DNA, 20 bp, synthetic construct
  misc_feature 1..20: note = Taqman probe 576BKV-TQ-FAM FAM
  misc_feature 20: note = conjugated to BHQ1
  source 1..20: mol_type = other DNA
ccaaaaagccaaaggaaccc

SEQ ID NO: 49 - DNA, 22 bp, synthetic construct
  misc_feature 1..22: note = GADPH forward primer
  source 1..22: mol_type = other DNA
acaactttggtatcgtggaagg

SEQ ID NO: 50 - DNA, 19 bp, synthetic construct
  misc_feature 1..19: note = GADPH reverse primer
  source 1..19: mol_type = other DNA
gccatcacgccacagtttc

SEQ ID NO: 51 - DNA, 23 bp, synthetic construct
  misc_feature 1..23: note = Tag forward primer
  source 1..23: mol_type = other DNA
gaggaggatgtaaaggtagctca

SEQ ID NO: 52 - DNA, 23 bp, synthetic construct
  misc_feature 1..23: note = Tag reverse primer
  source 1..23: mol_type = other DNA
actggcaaacatatcttcatggc

SEQ ID NO: 53 - DNA, 20 bp, synthetic construct
  misc_feature 1..20: note = VP1 forward primer
  source 1..20: mol_type = other DNA
tgcagggtcacaaaaagtgc

SEQ ID NO: 54 - DNA, 20 bp, synthetic construct
  misc_feature 1..20: note = VP1 reverse primer
  source 1..20: mol_type = other DNA
agcactccctgcatttccaa

SEQ ID NO: 55 - DNA, 39 bp, synthetic construct
  misc_feature 1..39: note = Donor site I, IV
  source 1..39: mol_type = other DNA
gaactggagtagctcagaggtttgtgctgattttcctct

SEQ ID NO: 56 - DNA, 39 bp, synthetic construct
  misc_feature 1..39: note = Donor site II, III
  source 1..39: mol_type = other DNA
gaactggaatagctcagaggtttgtgctgattttcctct

SEQ ID NO: 57 - DNA, 39 bp, synthetic construct
  misc_feature 1..39: note = Donor site consensus
  source 1..39: mol_type = other DNA
gaactggartagctcagaggtttgtgctgattttcctct

SEQ ID NO: 58 - DNA, 37 bp, synthetic construct
  misc_feature 1..37: note = Acceptor site I
  misc_feature 15: note = 2-methoxy ethyl modified ribonucleic acid
  source 1..37: mol_type = other DNA
taattatttktttttaggtgccaacctatggaacaga

SEQ ID NO: 59 - DNA, 40 bp, synthetic construct
  misc_feature 1..40: note = Acceptor site II
  source 1..40: mol_type = other DNA
taattatttttttkttataggtgccaacctatggaacaga

SEQ ID NO: 60 - DNA, 40 bp, synthetic construct
  misc_feature 1..40: note = Acceptor site III
  source 1..40: mol_type = other DNA
taattattttttttttataggtgccaacctatggaacaga

SEQ ID NO: 61 - DNA, 40 bp, synthetic construct
  misc_feature 1..40: note = Acceptor site IV, without gap
  source 1..40: mol_type = other DNA
taattatkttttttttataggtgccaacctatggaacaga

SEQ ID NO: 62 - DNA, 39 bp, synthetic construct
  misc_feature 1..39: note = Acceptor site IV, with gap
  source 1..39: mol_type = other DNA
taattakttttttttataggtgccaacctatggaacaga

SEQ ID NO: 63 - DNA, 39 bp, synthetic construct
  misc_feature 1..39: note = Acceptor site consensus, without gaps
  misc_feature 17: note = 2-methoxy ethyl modified ribonucleic acid
  source 1..39: mol_type = other DNA
taattatktttktttttaggtgccaacctatggaacaga

SEQ ID NO: 64 - DNA, 38 bp, synthetic construct
  misc_feature 1..38: note = Acceptor site consensus, with 1 gap
  misc_feature 16: note = 2-methoxy ethyl modified ribonucleic acid
  source 1..38: mol_type = other DNA
taattattttktttttaggtgccaacctatggaacaga

SEQ ID NO: 65 - DNA, 38 bp, synthetic construct
  misc_feature 1..38: note = Acceptor site consensus, with 1 gap
  misc_feature 16: note = 2-methoxy ethyl modified ribonucleic acid
  source 1..38: mol_type = other DNA
taattaktttktttttaggtgccaacctatggaacaga

SEQ ID NO: 66 - DNA, 37 bp, synthetic construct; identical to SEQ ID NO: 58
  misc_feature 1..37: note = Acceptor site consensus, with 2 gaps
  misc_feature 15: note = 2-methoxy ethyl modified ribonucleic acid
  source 1..37: mol_type = other DNA

SEQ ID NO: 67 - RNA, 20 nt, synthetic construct
  misc_feature 1..20: note = HYB_03 (native)
  source 1..20: mol_type = other RNA
cagcacaaacctctgagcta

SEQ ID NO: 68 - RNA, 20 nt, synthetic construct
  misc_feature 1..20: note = HYB_03 (2O-Me)
  modified_base 1..19: mod_base = OTHER; note = phosphorothioate linkage
  modified_base 1..20: mod_base = OTHER; note = 2'-O-methyl
  source 1..20: mol_type = other RNA
cagcacaaacctctgagcta

SEQ ID NO: 69 - RNA, 20 nt, synthetic construct
  misc_feature 1..20: note = HYB_03 (MOE)
  modified_base 1..19: mod_base = OTHER; note = phosphorothioate linkage
  modified_base 1..20: mod_base = OTHER; note = 2'-methoxy-ethyl
  source 1..20: mol_type = other RNA
cagcacaaacctctgagcta

SEQ ID NO: 70 - DNA, 23 bp, synthetic construct
  source 1..23: mol_type = other DNA
gaggaggatgtaaaggtagctca

SEQ ID NO: 71 - DNA, 23 bp, synthetic construct
  source 1..23: mol_type = other DNA
actggcaaacatatcttcatggc

SEQ ID NO: 72 - DNA, 20 bp, synthetic construct
  source 1..20: mol_type = other DNA
tgcagggtcacaaaaagtgc

SEQ ID NO: 73 - DNA, 20 bp, synthetic construct
  source 1..20: mol_type = other DNA
agcactccctgcatttccaa

SEQ ID NO: 74 - DNA, 22 bp, synthetic construct
  source 1..22: mol_type = other DNA
acaactttggtatcgtggaagg

SEQ ID NO: 75 - DNA, 19 bp, synthetic construct
  source 1..19: mol_type = other DNA
gccatcacgccacagtttc

SEQ ID NO: 76 - RNA, 20 nt, synthetic construct
  source 1..20: mol_type = other RNA
aaatcagcacaaacctctga

SEQ ID NO: 77 - RNA, 20 nt, synthetic construct
  source 1..20: mol_type = other RNA
gaaaatcagcacaaacctct